(19) United States
(12) Patent Application Publication
(10) Pub. No.: US 2026/0256825 A1
(43) Pub. Date: Sep. 3, 2026

(54) Metabolomic Improvements Using Resistant Starch Supplementation

(71) Applicant: McPharma Biotech Inc., Carberry (CA)

(72) Inventors: Derek McLaren, Carberry (CA); Earl McLaren, Carberry (CA)

(21) Appl. No.: 18/878,403

(22) PCT Filed: Jun. 26, 2023
(86) PCT No.: PCT/CA2023/050884

§ 371 Date: Dec. 23, 2024

Related U.S. Application Data
(60) Provisional application No. 63/358,194, filed on Jul. 4, 2022.

(51) Int. Cl. A61K 31/718; A61K 36/81

(52) U.S. Cl. CPC A61K 31/718; A61K 36/81

(57) ABSTRACT

Resistant potato starch (RPS) is a form of dietary fiber that is valued for improving insulin sensitivity and promoting 2024/007072 increases in Bifidobacterium in the intestines. RPS administration in human trials produces unexpected beneficial changes in the abundance of several metabolites related to memory and cognitive function, bile acid metabolism, immune function, collagen metabolism, polyamine levels, mitochondrial function, oxidative stress, athletic performance and male health, and fatty acid hydroxylation. The diverse categories of metabolites influenced by RPS reveal numerous underappreciated benefits to consuming this form of dietary fiber.

### PRIOR APPLICATION INFORMATION

The instant application claims the benefit of U.S. Provisional Patent Application 63/358,194, filed Jul. 4, 2022, entitled “METABOLOMIC IMPROVEMENTS USING RESISTANT STARCH SUPPLEMENTATION”, the entire contents of which are incorporated herein by reference for all purposes.

### BACKGROUND OF THE INVENTION

Resistant starch (RS) is “the sum of all starch and starch degradation products that, on average, reach the human large intestine” [1]. RS is therefore a form of dietary fibre, which has been recognized by regulatory bodies. (for example, https://www.canada.ca/en/health-canada/services/publications/food-nutrition/list-reviewed-accepted-dietary-fibres.html) RS levels in foods customarily consumed by Americans are low [2], in contrast to historical diets that contained 20 g or more of RS per day [3]. Experts suggest that restoring dietary intake of RS to levels of at least 15 g per day is required to improve gastrointestinal health and promote insulin sensitivity [2]. However, the relationship between RS and broader aspects of human health have not been explored.

The ‘metabolome’ refers to “the repertoire of biochemicals (or small molecules) present in cells, tissues, and body fluids” [4]. It is believed that “a person's metabolic state provides a close representation of that individual's overall health status” and that this metabolic state reflects the person's genome, and external influences including the diet, environmental factors, and the gut microbiota [4]. Changes in metabolites can influence human physiology by promoting or inhibiting various cellular activities or biochemical processes, leading to the investigation of dietary inputs that can positively improve a person's health via discrete metabolomic changes or changes affecting suites of metabolites.

RS supplementation or the consumption of RS-rich foods is associated with increases in the microbiome-derived metabolites called short chain fatty acids (SCFAs), most notably butyrate [5]. We previously found significant increases in the abundance of butyrate in stool samples from elderly individuals consuming 30 g of resistant potato starch (RPS) per day [6]. Butyrate is beneficial for numerous aspects of cellular and host health, including the prevention of cancer and the promotion of normal epithelial cell function [7]. While some of these benefits involve local activity of butyrate in the colon, the systemic effects associated with butyrate are poorly correlated with butyrate levels in stool, which represent butyrate produced in the intestines that was not absorbed by colonocytes or the host body. Supplementation with butyrate has not provided benefits to patients with metabolic syndrome, which has been attributed to the rapid clearance and poor availability of systemic butyrate [8].

In the present study, we sought to evaluate the effect of RS on SCFA levels in serum by measuring the effects of different doses at different durations in two clinical trials. In addition, we measured other small organic acids, medium and long chain fatty acids, amines and amino acids, carnitines, and bile acids in these serum samples using targeted panel assays. Surprisingly, we found that RPS had no significant effects on butyrate or other SCFAs in serum. However, we detected significant changes in serum levels of several different metabolites, suggesting that RPS supplementation may influence host physiology in more ways than were previously known.

### SUMMARY OF THE INVENTION

According to an aspect of the invention, there is provided a method of modifying levels of a microbially-influenced metabolite in human serum generated by resistant potato starch fermentation in the human's gut comprising administering to said human an effective amount of a suitable resistant potato starch on a dosage regimen.

According to an aspect of the invention, there is provided a method of increasing circulating neurosteroid pregnenolone sulfate levels in an individual in need of such treatment comprising administering to said individual an effective amount of a suitable resistant potato starch on a dosage regimen.

In another aspect of the invention, there is provided a method of increasing circulating levels of 3-beta-hydroxy-5-cholestenoic acid in an individual in need of such treatment comprising administering to said individual an effective amount of a suitable resistant potato starch on a dosage regimen.

In another aspect of the invention, there is provided a method of decreasing circulating levels of glycine and/or taurine conjugated bile acids in an individual in need of such treatment comprising administering to said individual an effective amount of a suitable resistant potato starch on a dosage regimen.

In another aspect of the invention, there is provided a method of decreasing circulating levels of bile acids in an individual in need of such treatment comprising administering to said individual an effective amount of a suitable resistant potato starch on a dosage regimen.

In another aspect of the invention, there is provided a method of decreasing bacterial bile salt hydrolase activity in the gut of an individual in need of such treatment comprising administering to said individual an effective amount of a suitable resistant potato starch on a dosage regimen.

In another aspect of the invention, there is provided a method of decreasing the circulating levels of 3-hydroxykynurenine in an individual in need of such treatment comprising administering to said individual an effective amount of a suitable resistant potato starch on a dosage regimen.

In another aspect of the invention, there is provided a method of decreasing circulating levels of the amine histamine in an individual in need of such treatment comprising administering to said individual an effective amount of a suitable resistant potato starch on a dosage regimen.

In another aspect of the invention, there is provided a method of decreasing circulating levels of the collagen breakdown product 5-hydroxylysine in an individual in need of such treatment comprising administering to said individual an effective amount of a suitable resistant potato starch on a dosage regimen.

In another aspect of the invention, there is provided a method of decreasing circulating levels of the collagen breakdown product 4-hydroxyproline in an individual in need of such treatment comprising administering to said individual an effective amount of a suitable resistant potato starch on a dosage regimen.

In another aspect of the invention, there is provided a method of preventing collagen breakdown in an individual in need of such treatment comprising administering to said individual an effective amount of a suitable resistant potato starch on a dosage regimen.

In another aspect of the invention, there is provided a method of decreasing circulating levels of the polyamine N-acetyl putrescine in an individual in need of such treatment comprising administering to said individual an effective amount of a suitable resistant potato starch on a dosage regimen.

In another aspect of the invention, there is provided a method of decreasing circulating levels of the polyamine N-acetyl spermidine in an individual in need of such treatment comprising administering to said individual an effective amount of a suitable resistant potato starch on a dosage regimen.

In another aspect of the invention, there is provided a method of enhancing circulating levels of the polyamines in an individual in need of such treatment comprising administering to said individual an effective amount of a suitable resistant potato starch on a dosage regimen.

In another aspect of the invention, there is provided a method of increasing circulating acylcarnitine levels in an individual in need of such treatment comprising administering to said individual an effective amount of a suitable resistant potato starch on a dosage regimen.

In another aspect of the invention, there is provided a method of decreasing circulating short-and medium-chain carnitines to free carnitine ratios in an individual in need of such treatment comprising administering to said individual an effective amount of a suitable resistant potato starch on a dosage regimen.

In another aspect of the invention, there is provided a method of enhancing mitochondrial β-oxidation in an individual in need of such treatment comprising administering to said individual an effective amount of a suitable resistant potato starch on a dosage regimen.

In another aspect of the invention, there is provided a method of increasing circulating levels of propionyl carnitine in an individual in need of such treatment comprising administering to said individual an effective amount of a suitable resistant potato starch on a dosage regimen.

In another aspect of the invention, there is provided a method of increasing the circulating levels of serotonin by decreasing the of 5-hydroxyindoleacetic acid to serotonin ratio in an individual in need of such treatment comprising administering to said individual an effective amount of a suitable resistant potato starch on a dosage regimen.

In another aspect of the invention, there is provided a method of increasing circulating levels of Coenzyme Q10 by increasing the Coenzyme Q10 to phenylalanine ratio in an individual in need of such treatment comprising administering to said individual an effective amount of a suitable resistant potato starch on a dosage regimen.

In another aspect of the invention, there is provided a method of enhancing mitochondrial function by decreasing circulating short-and medium-chain to free carnitine ratios or increasing the Coenzyme Q10 to phenylalanine ratio in an individual in need of such treatment comprising administering to said individual an effective amount of a suitable resistant potato starch on a dosage regimen.

According to another aspect of the invention, there is provided a method of decreasing circulating levels of hydroxydecanoic acid in an individual in need of such treatment comprising administering to said individual an effective amount of a suitable resistant potato starch on a dosage regimen.

In some embodiments of the invention, the dosage regimen is administering resistant potato starch daily for a period of 1-12 weeks. In this context, “daily” does not necessarily mean “every day”, but may mean for example 19 out of 20 days, 9 out of 10 days, 17 out of 20 days or 8 out of 10 days.

As discussed herein, the effective amount may be, for example, 0.5 to 40 g, or 0.5 to 30 g, or 0.5 to 7 g, or 0.5 to 3.5 g of resistant starch.

### BRIEF DESCRIPTION OF THE DRAWINGS

FIG. 1A. Serum was collected at baseline and after one and four weeks of supplementation. Serum levels of pregnenolone sulfate are significantly greater after four weeks of supplementation with 3.5 g and 7 g of resistant potato starch (RPS). (3.5 g=3.5 g/day RPS, 7 g=7 g/day RPS, Placebo=fully digestible corn starch; *p<0.05, two tailed t-test, error bars=SEM)

FIG. 1B. Serum levels of 3-beta-hydroxy-5-cholestenoic acid are significantly greater after one or four weeks of supplementation with 7 g RPS or after 4 weeks with 3.5 g RPS. (*p<0.05, two tailed t-test, error bars=SEM)

FIG. 2A. Serum was collected at baseline and after one and four weeks of supplementation. Serum levels of glyco-a-muricholic acid are significantly reduced after one week of supplementation with 3.5 g RPS. (*p<0.05, two tailed t-test, error bars=SEM)

FIG. 2B. Serum was collected at baseline and after 12 weeks of supplementation. Serum levels of glyco-α-muricholic acid are significantly reduced in the pooled cohorts after 12 weeks of supplementation with 30 g RPS. (MID=middle aged, ELD=elderly, Pooled=MID plus ELD cohorts; * p<0.05, two tailed t-test, error bars=SEM)

FIG. 2C. Serum levels of glycohyocholic acid are significantly reduced after one week of supplementation with 3.5 g RPS. (*p<0.05, two tailed t-test, error bars=SEM)

FIG. 2D. Serum levels of glycohyocholic acid are significantly reduced in the elderly (ELD) after 12 weeks of supplementation with 30 g RPS. (*p<0.05, two tailed t-test, error bars=SEM)

FIG. 2E. Serum levels of glycoursodeoxycholic acid are significantly reduced in the ELD cohort after 12 weeks of supplementation with 30 g RPS. (*p<0.05, two tailed t-test, error bars=SEM)

FIG. 3A. Serum was collected at baseline and after one and four weeks of supplementation. Serum levels of taurohyocholic acid are significantly reduced after one week of supplementation with 3.5 g RPS. (*p<0.05, two tailed t-test, error bars=SEM)

FIG. 3B. Serum levels of tauro-w-muricholic acid are significantly reduced after one week of supplementation with 3.5 g RPS. (*p<0.05, two tailed t-test, error bars=SEM)

FIG. 3C. Serum levels of tauroursodeoxycholic acid are significantly reduced after one week of supplementation with 3.5 g RPS. (*p<0.05, two tailed t-test, error bars=SEM)

FIG. 3D. Serum was collected at baseline and after 12 weeks of supplementation. Serum levels of tauroursodeoxycholic acid are significantly reduced in the ELD cohort after 12 weeks of supplementation with 30 g RPS. (*p<0.05, two tailed t-test, error bars=SEM)

FIG. 3E. Serum levels of taurochenodeoxycholic acid are significantly reduced after one week of supplementation with 3.5 g RPS. (*p<0.05, two tailed t-test, error bars=SEM)

FIG. 3F. Serum levels of taurochenodeoxycholic acid 3-sulfate are significantly reduced in the pooled cohorts after 12 weeks of supplementation with 30 g RPS. (*p<0.05, two tailed t-test, error bars=SEM)

FIG. 3G. Ratios of Glyco-and tauro-conjugated primary and secondary bile acids to unconjugated primary and secondary bile acids in serum were significantly reduced in the 3.5 g RPS treatment group compared to those in the placebo. (*p <0.05, two-way ANOVA, error bars=SEM)

FIG. 4A. Serum was collected at baseline and after 12 weeks of supplementation. Serum levels of muricholic acid are significantly reduced in the pooled cohorts after 12 weeks of supplementation with 30 g RPS. (*p<0.05, two tailed t-test, error bars=SEM)

FIG. 4B. Serum levels of ursodeoxycholic acid 3-glucuronide are significantly reduced in the pooled cohorts after 12 weeks of supplementation with 30 g RPS. (*p<0.05, two tailed t-test, error bars=SEM)

FIG. 5A. Serum was collected at baseline and after one and four weeks of supplementation. Serum levels of 5-hydroxylysine are significantly reduced after one week of supplementation with 3.5 g RPS. (*p<0.05, two tailed t-test, error bars=SEM)

FIG. 5B. Serum levels of histamine are significantly reduced after one week of supplementation with 3.5g RPS. (*p<0.05, two tailed t-test, error bars=SEM)

FIG. 5C. Serum levels of 4-hydroxyproline are significantly reduced in participants consuming 3.5 g/day RPS compared to levels in people consuming the placebo. (*p<0.05, two-way ANOVA, error bars=SEM)

FIG. 6A. Serum was collected at baseline and after one and four weeks of supplementation. Serum levels of N-acetyl putrescine are significantly reduced after one week of supplementation with 3.5 g RPS. (*p<0.05, two tailed t-test, error bars=SEM)

FIG. 6B. Serum was collected at baseline and after 12 weeks of supplementation. Serum levels of N-acetyl putrescine are significantly reduced in the ELD cohort after 12 weeks of supplementation with 30 g RPS. (*p<0.05, two tailed t-test, error bars=SEM)

FIG. 6C. Serum levels of N-acetyl spermidine are significantly reduced after four weeks of supplementation with 3.5 g and 7 g RPS. (*p<0.05, two tailed t-test, error bars=SEM)

FIG. 6D. Serum levels of N-acetyl putrescine are significantly reduced in the middle aged (MID) and pooled cohorts after 12 weeks of supplementation with 30 g RPS. (*p<0.05, two tailed t-test, error bars=SEM)

FIG. 7A. Serum was collected at baseline and after 12 weeks of supplementation. Serum levels of propionyl carnitine are significantly increased in the MID and pooled cohorts after 12 weeks of supplementation with 30 g RPS. (*p<0.05, two tailed t-test, error bars=SEM)

FIG. 7B. Serum levels of heptanoyl carnitine are significantly increased in the pooled cohorts after 12 weeks of supplementation with 30 g RPS. (*p<0.05, two tailed t-test, error bars=SEM)

FIG. 8A. Serum was collected at baseline and after one and four weeks of supplementation. Serum levels of hydroxydecanoic acid are significantly reduced after four weeks of supplementation with 7 g RPS. (*p<0.05, two tailed t-test, error bars=SEM)

FIG. 8B. Serum was collected at baseline and after 12 weeks of supplementation. Serum levels of hydroxydecanoic acid are significantly reduced in the ELD cohort after 12 weeks of supplementation with 30 g RPS. (*p<0.05, two tailed t-test, error bars=SEM)

FIG. 9A. Serotonin levels tend to increase but treatment effects are not significantly different. (p>0.05, treatment effect, ANOVA, error bars=SEM)

FIG. 9B. The serotonin to tryptophan ratio was not significantly different between treatments. (p>0.05, treatment effect, ANOVA, error bars=SEM)

FIG. 9C. The ratio of serotonin degradation product 5-hydroxyindoleacetic acid to serotonin, reflecting monoamine oxidase activity, was significantly reduced in the 3.5 g RPS treatment arm. (*p<0.05, ANOVA, error bars=SEM)

FIG. 10A. Coenzyme Q10 levels significantly increased in both placebo (p=0.0078, one way ANOVA, error bars=SEM), and 3.5 g RPS treatment arms (p=0.0079, one way ANOVA, error bars=SEM), but were not significantly different from each other (p=0.5729, two-way ANOVA, error bars=SEM).

FIG. 10B. Coenzyme Q10 is synthesized from amino acids tyrosine or phenylalanine, to produce 4-hydroxybenzoate, then benzoquinone, and from Acetyl-CoA condensation to mevalonate to form a polyisoprenyl chain.

FIG. 10C. Tyrosine levels were significantly reduced in the 3.5 g RPS treatment arm compared to the placebo. (*p<0.05, ANOVA, error bars=SEM)

FIG. 10D. Phenylalanine levels were significantly reduced in the 3.5 g RPS treatment arm compared to the placebo. (*p<0.05, ANOVA, error bars =SEM)

FIG. 10E. Changes in the ratio of Coenzyme Q10 to tyrosine were not significantly different between treatment arms. (p=0.08), ANOVA, error bars=SEM).

FIG. 10F. Changes in the ratio of Coenzyme Q10 to phenylalanine were significantly higher in the 3.5 g RPS arm compared to the placebo. (*p<0.05, ANOVA, error bars=SEM)

FIG. 11A. The ratio of short-and medium-chain carnitines to free carnitine was significantly reduced in the 3.5 g RPS arm compared to the placebo. (*p<0.05, ANOVA, error bars=SEM)

FIG. 11B. The ratio of long-chain carnitines to free carnitine was not significantly affected by treatment. (p>0.05, ANOVA, error bars=SEM)

FIG. 11C. There was no effect of treatment on the abundance of dicarboxyl carnitines. (p>0.05, ANOVA, error bars=SEM)

FIG. 12A. Levels of the tryptophan metabolite kynurenine were significantly elevated in the placebo arm compared to the 3.5 g RPS arm. (*p<0.05, ANOVA, error bars=SEM)

FIG. 12B. Levels of 3-hydroxykynurenine were significantly decreased in the 3.5 g RPS treatment arm compared to the placebo. (*p<0.05, ANOVA, error bars=SEM)

FIG. 12C. The ratio of 3-hydroxykynurenine to kynurenine, reflecting kynurenine monooxygenase activity, was not significantly affected by treatment. (p>0.05, ANOVA, error bars=SEM)

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “treating”, in all of its grammatical forms, does not necessarily require the “curing” of the disease, disorder, episode, occurrence or incident, but rather refers to at least one of the following: lessening the frequency and/or duration and/or severity of feelings of illness and/or one or more symptoms associated with the disease, disorder, episode, occurrence or incident.

As used herein, “circulating levels” refers to the levels of the compound(s) circulating in the blood of the patient or subject. Preferably, the patient or subject is a human.

We investigated the effects of resistant potato starch (Solnul™) in two separate clinical trials. MSP Starch Products Inc manufactures Solnul™, an unmodified RS type 2 that is a food grade quality potato starch for human and animal food applications. While Solnul™, which contains 60% RS by AOAC 2002.02 methods, is used in the trials and experiments discussed herein, it is important to note that as discussed herein, another suitable unmodified potato starch or resistant potato starch (RS type 2) comprising at least 50% resistant starch or at least 55% resistant starch or at least 60% resistant starch on an ‘as is’basis and meeting USP criteria for potato starch may be used.

As discussed herein, we demonstrate that supplementation with resistant potato starch (RPS) modulated the levels of several metabolites in serum. Specifically, RPS increased serotonin by decreasing the 5-hydroxyindoleacetic acid to serotonin ratio, the neurosteroid pregnenolone sulfate and the gamma secretase inhibitor 3-beta-hyrdoxy-5-cholestenoic acid, as well as levels of carnitines propionyl carnitine and heptanoyl carnitine, and Coenzyme Q10 by increasing the Coenzyme Q10 to phenylalanine ratio.

Furthermore, RPS decreased serum levels of bile acids, including bile acids conjugated to glycine and taurine, as well as the collagen degradation products 4-hydroxyproline and 5-hydroxylysine, the inflammation-associated amine histamine, polyamines N-acetyl putrescine and N-acetyl spermidine, the ratio of short-and medium-chain carnitines to free carnitines, neurotoxic metabolite 3-hydroxykynurenine, and hydroxydecanoic acid.

The first clinical trial was a prospective, randomized, double-blinded, placebo-controlled study conducted in Winnipeg, MB, Canada by Source Nutraceutical (Winnipeg, MB) and Cliantha Research (formerly Hill Top Research; Ahmedabad, GJ, India) from Apr 2019 to Oct 2019. A formal power analysis was not conducted as the study was designed to replicate a previous clinical trial [9] [6]. Participants were segregated by age into sub-groups (30-50 years; MID or >70 years; ELD) and then assigned to placebo or study product based on the randomization list generated by Karmic Life Sciences (Mumbai, MH, India). Trial participants, clinical investigators, outcome assessors, and data analysts were blinded to which treatment participants were assigned. All information collected for the purpose of the study was kept in a locked and secured area. All information collected and sent for statistical analyses only had a study number and no participant identifiers. All participant identifiers were treated in confidence and in accordance with the Personal Health Information Act of Manitoba.

The study was explained verbally and in written format to eligible participants, and all participants provided written informed consent. Participants were informed that they could request to withdraw from the clinical study at any time without adverse affects. Exclusion criteria included: Pregnancy, planned pregnancy, or breastfeeding during the study, Crohn's disease or other inflammatory bowel disease, individuals with systemic lupus erythematosus or on cancer chemotherapy, pre-diabetes or diabetes, thyroid disease, renal disease, hepatic disease, previous gastrointestinal surgery (intestinal resection, gastric bypass, colorectal surgery), individuals consuming probiotics (including potential probiotic foods like yogurt), individuals on antibiotics at time of recruitment or on antibiotics within the previous five weeks, individuals experiencing dysphagia, subjects using additional fiber supplements, subjects allergic to potato or corn, and individuals on digestants, emetics, anti-emetics, medications for acid peptic disease or taking antacids. Female participants took pregnancy tests at each visit to ensure they were not pregnant. There were no changes to the normal daily diet consumed by participants other than the requirement that they did not consume probiotic-containing products. Subjects were screened to determine their eligibility according to the inclusion/exclusion criteria outlined above and blood samples were screened for abnormalities in urea, creatinine, aspartate transaminase, alanine transferase, alkaline phosphatase, and creatine kinase (Dynacare, Brampton, ON). After screening, a qualified investigator reviewed the subject health information for their inclusion in the study.

All eligible study participants were enrolled in a two-week run-in period, during which time they ingested 30 g of placebo once daily to acclimatize all subjects to taking the supplement. Subjects were advised to mix the product in 250 ml of non-heated fluid or non-heated semi-solid food, and those taking medication were advised to take the product either 2 hours before or 2 hours after taking the medication. On all study visits, subjects had their height, weight, Body Mass Index (BMI), waist and hip circumference measurements taken, and blood pressure checked. Following the run-in period, subjects were randomized to consume 30 g per day of RPS or fully digestible corn starch placebo (each individually packaged in 30 g amounts) for 12 weeks.

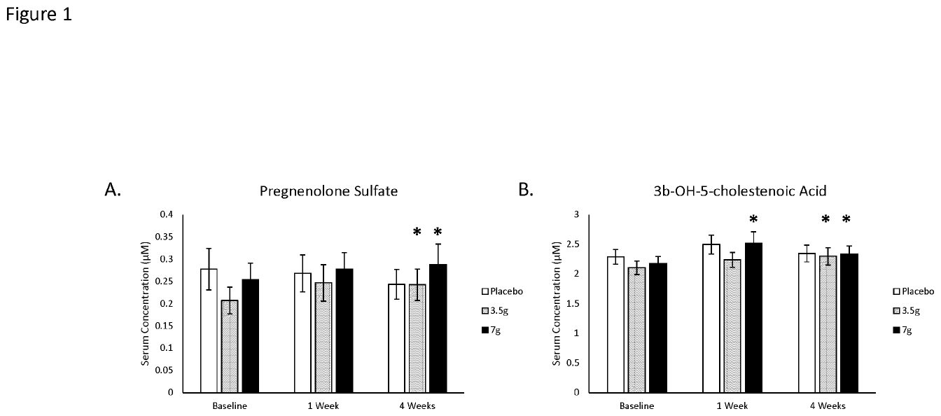

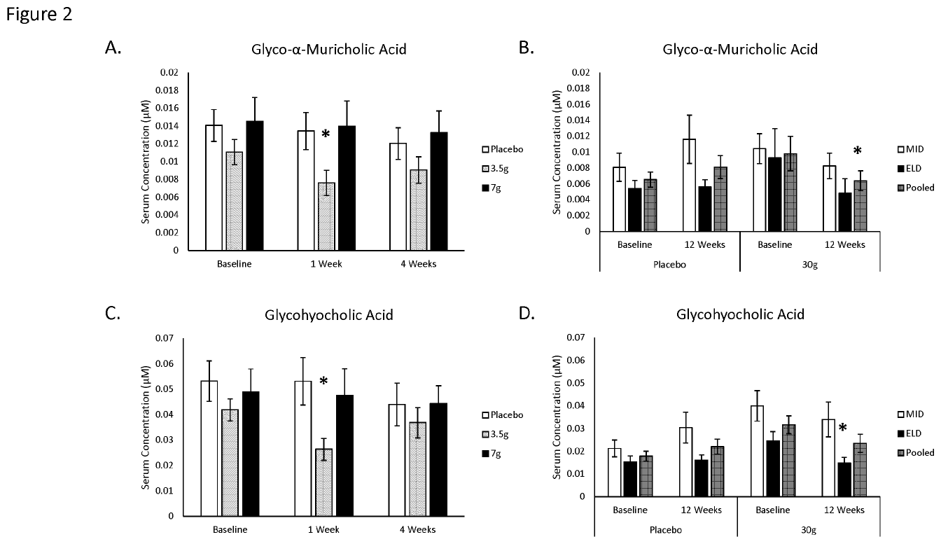

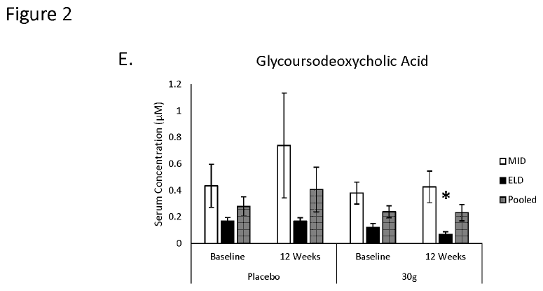

Daily records of antibiotic use, hospital admissions, and medical visits were collected. Fasting serum samples were collected at baseline and the end of the 12-week intervention and were stored at −80° C. (MRM Proteomics, Montreal, QC) before mass spectroscopy analysis (The Metabolomics Innovation Centre/The UVic-Genome BC Proteomics Centre, Victoria, BC). In total, data was analyzed for 81 participants (FIGS. 1 and 2), 44 who received RPS (20 MID, 24 ELD) and 37 who received placebo (15 MID, 22 ELD).

In the second clinical trial, participants were generally healthy adult males and females between 18-69 years of age with a body mass index (BMI) of 18.0 to ≤34.9 kg/m². Enrolled participants agreed to not use any new vitamin, mineral or dietary supplement product until after the study completion and to not take any vitamins, minerals, or dietary supplements 14 days prior to Visit 2 (Randomization) until the completion of Visit 4, since consumption of these products may bias the results for the test product. Individuals with a BMI over 34.9 kg/m² were excluded as their health and any related metabolic changes may impact the results of this study. For similar health reasons, any individuals with a diagnosis of irritable bowel syndrome, dyspepsia, significant gastrointestinal disorders, or other major diseases were excluded. A formal sample size calculation was not performed. The sample size of 25 subjects per study group is consistent with our previous clinical investigation of RS [9] [6].

A total of 98 participants from Guelph, ON and the surrounding area were screened for eligibility to obtain the required sample size of 75 participants (25 participants per study arm) who were enrolled in the study, which occurred from Oct. 20, 2019 to Jan. 6, 2020. The identity of the study intervention was blinded to the study staff and participants. The study included a screening visit from 30 days up to 14 days prior to randomization, a run in-period of 14 to 17 days prior to randomization, a baseline visit (Day 0) during which the randomization was performed, and serum collected, and 2 subsequent study visits at Weeks 1 and 4, respectively, where serum was collected.

During the baseline visit (Day 0), the participants were randomized to receive one of three study interventions as indicated by the randomization scheme: high dose (7 g resistant potato starch (RPS)), low dose (3.5 g RPS combined with 3.5 g digestible corn starch), or placebo (7 g digestible corn starch). The randomization scheme was generated by Nutrasource using SAS 9.4 PROC PLAN on 22 OCT 19: 15:44:40 with Seed Number: 1887363180. 75 subjects were randomized into 25 blocks with each block containing 3 subjects. The first dose of study intervention was demonstrated and administered by mixing the product in approximately 125 mL of cool or room temperature water and immediately drinking before the investigational product settled. Participants were instructed to consume the investigational product in the morning. A 31-day supply of the study intervention was provided to the study participants during the baseline visit. At Visit 3 (Week 1), unused study interventions/empty packaging were collected, and compliance was calculated. At Visit 4 (Week 4, the final study visit), unused study products/empty packaging were collected, and compliance was calculated.

Participants were instructed that for Visits 3 and 4, they should return all sachets they were given and to not throw away any open or unopened sachets. Compliance was calculated based on the amount of study product consumed compared to the total amount of study product expected to have been consumed for the given duration. Compliance for this study was considered acceptable if participants consumed an average of ≥80% of the study product for the given duration. One participant per treatment arm discontinued the study before Visit 4, and 2 participants in RPS high dose arm were excluded due to non-compliance (<80% product consumption) and use of study-prohibited medication. Safety profiles were based on the safety analysis set (SAF; n=75), while the full analysis set (FAS; n=72) included participants who received at least one dose of study product and had at least one outcome assessment after dosing and the per protocol population (PP; n=70) included only those who completed the study with overall compliance with study parameters. Serum samples were stored at −80° C. (MRM Proteomics, Montreal, QC) before mass spectroscopy analysis (The Metabolomics Innovation Centre/The UVic-Genome BC Proteomics Centre, Victoria, BC).

Mass spectroscopy analysis of metabolites in serum was completed by The Metabolomics Innovation Centre (Victoria, BC). UPLC-MRM/MS was carried out on an Agilent 1290 UHPLC (Agilent Technologies, Santa Clara, CA) coupled to a Sciex 4000 QTRAP mass spectrometer (AB Sciex, Framingham, MA) operated in the multiple-reaction monitoring mode with negative-ion detection. Internal standard solutions were prepared in MeOH-acetonitrile and serially diluted with water. Serum samples were thawed at room temperature, aliquots mixed with the internal standard solution, vortexed, and sonicated in a water bath, before centrifugal clarification. Aliquots of supernatant were mixed with water to create sample solutions. A pooled sample of 10 randomly selected aliquots was also prepared as the QC sample solution, which was injected after every 20 samples. UPLC-MRM/MS data were acquired with Sciex Analyst software and batch processed with Sciex MultiQuant software (AB Sciex). Linear calibration curves of metabolites were constructed using standard solution concentrations for each analyte with an appropriate concentration range versus analyte-to-internal standard peak area ratios. Concentrations detected in each sample were calculated by interpolating the calibration curves with the analyte-to-internal standard peak area ratios measured from each sample.

Baseline values for metabolites were compared to one week, four weeks (second trial), or twelve weeks (first trial) using Student's t-Test and p values <0.05 were considered significant unless t-Tests for changes in placebo arms also generated p values <0.05 with the same directionality as the RPS treatments in either study (i.e., Placebo treatment did not also produce a significant effect on the metabolite).

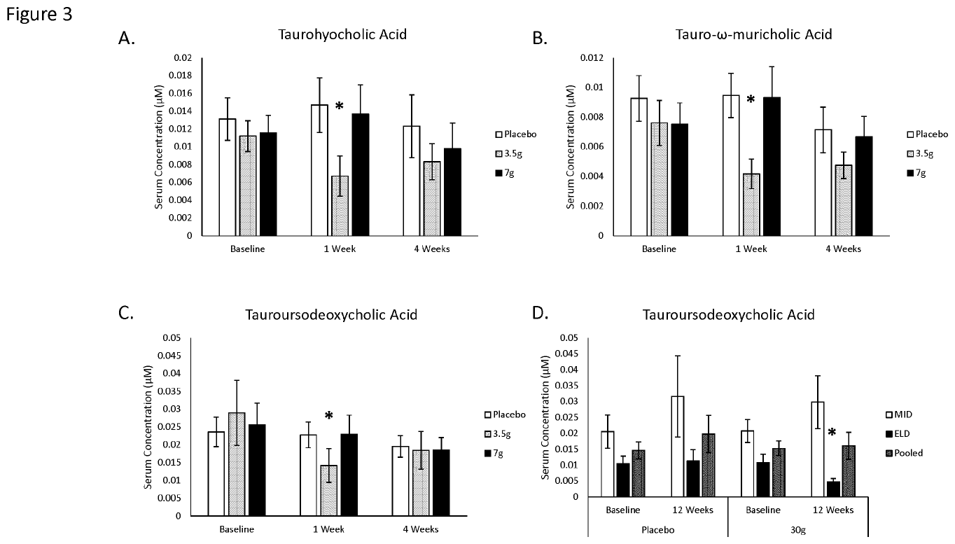

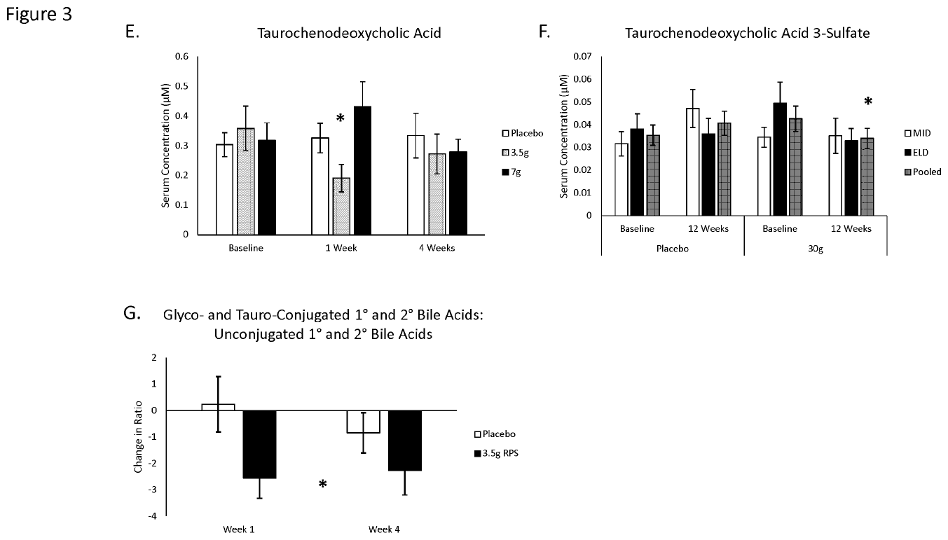

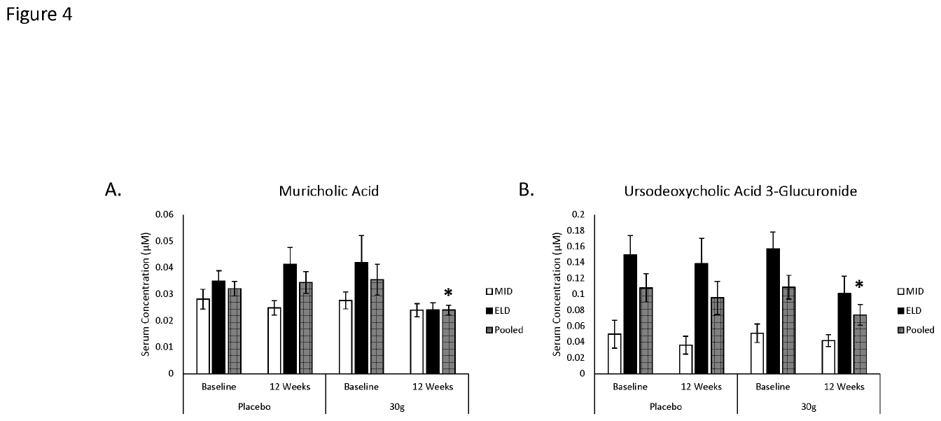

RPS led to significant increases in the neurosteroid pregnenolone sulfate (FIG. 1A) and the gamma secretase inhibitor 3-beta-hydroxy-5-cholestenoic acid (FIG. 1B) at both 3.5 g and 7 g per day doses. RPS supplementation significantly decreased glycine conjugated bile acids glycol-alpha-muricholic acid (FIG. 2A) and glycohyocholic acid (FIG. 2B) at 3.5 g per day, and 30 g/day of RPS significantly decreased glycol-alpha-muricholic acid in the pooled cohorts (FIG. 2B), and glycohyocholic acid (FIG. 2D), and glycoursodeoxycholic acid in the ELD cohort (FIG. 2E). Similarly, RPS supplementation significantly decreased taurine conjugated bile acids taurohyocholic acid (FIG. 3A), tauro-w-muricholic acid (FIG. 3B), tauroursodeoxycholic acid (FIG. 3C), and taurochenodeoxycholic acid (FIG. 3E) at 3.5 g per day. At 30 g/day, RPS significantly decreased tauroursodeoxycholic acid in the ELD cohort (FIG. 3D) and taurochenodeoxycholic acid 3-sulfate in the pooled cohorts (FIG. 3F). Comparing the ratio of glyco-and tauro-conjugated primary and secondary bile acids to unconjugated primary and secondary bile acids also demonstrated a significant reduction in the 3.5 g/day treatment arm compared to those in the placebo (FIG. 3G). The 30 g/day dose also significantly decreased muricholic acid (FIG. 4A) and ursodeoxycholic acid 3-glucuronide (FIG. 4B) in the pooled cohorts.

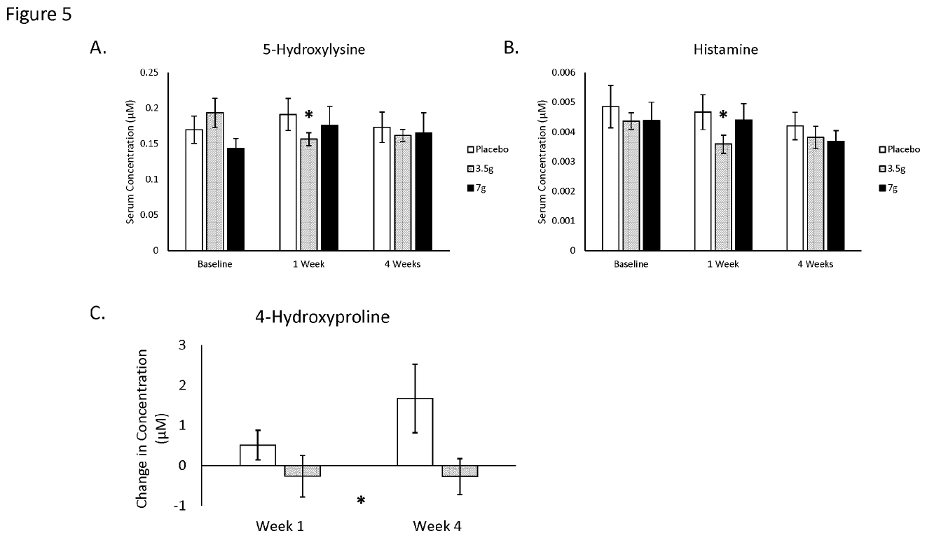

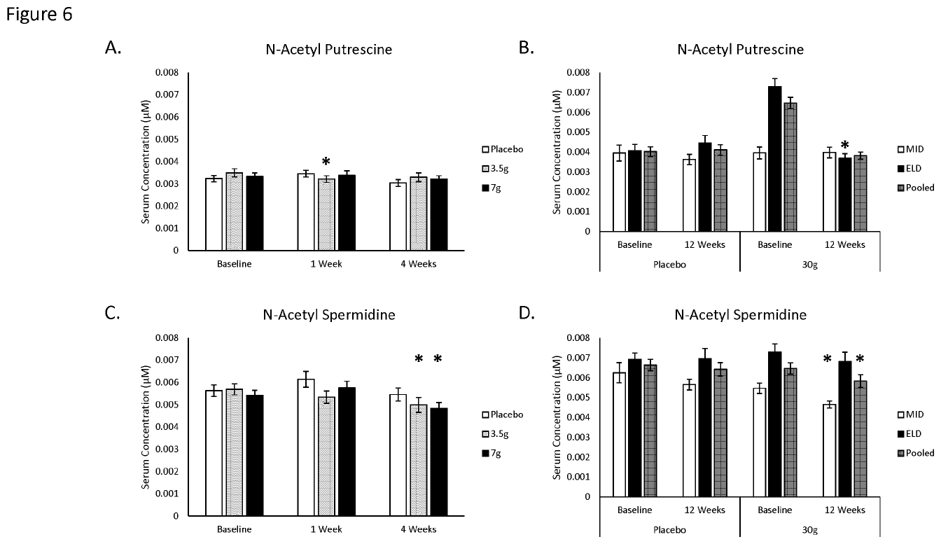

Participants consuming 3.5 g per day RPS experienced significant reductions in 5-hydroxylysine (FIG. 5A), histamine (FIG. 5B), and 4-hydroxyproline (FIG. 5C). Polyamine metabolism was affected across all RPS doses: N-acetyl putrescine was significantly decreased at 3.5 g (FIG. 6A) and at 30 g in the ELD cohort (FIG. 6B). N-acetyl spermidine was significantly reduced at 3.5 g and 7 g doses (FIG. 6C), as well as at 30 g in the middle aged (MID) and pooled cohorts (FIG. 6D).

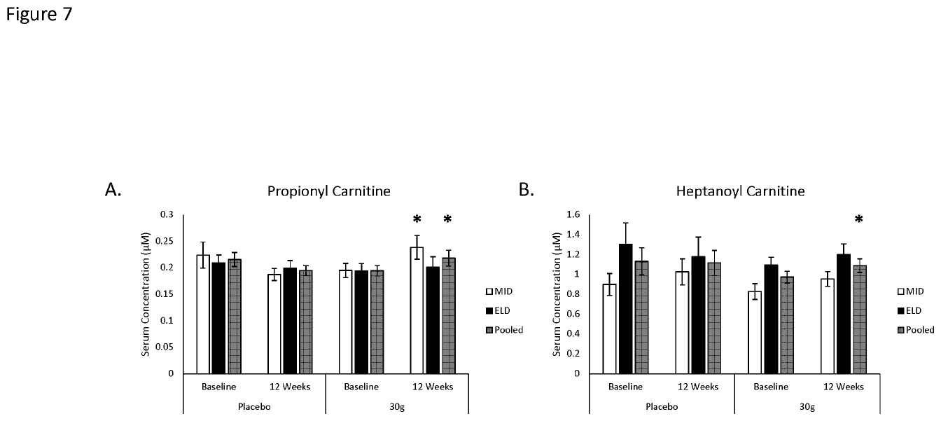

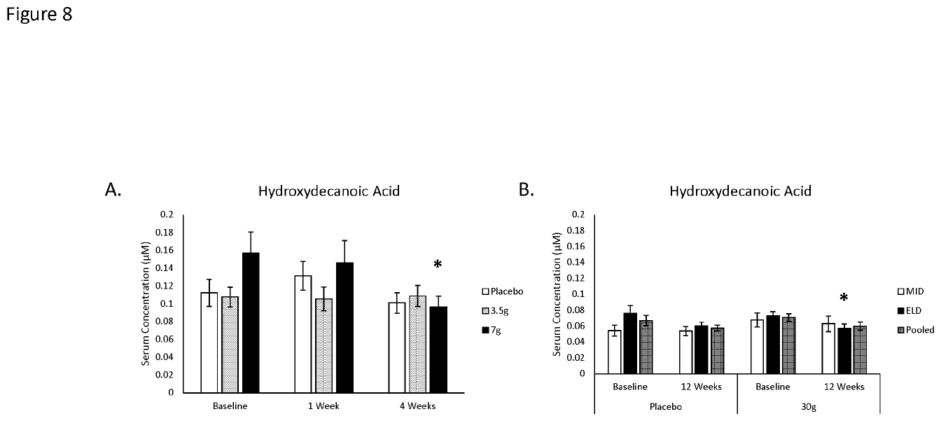

Acylcarnitine metabolism was significantly affected by 30 g doses of RPS. Propionyl carnitine increased in the MID and pooled populations (FIG. 7A) while heptanoyl carnitine increased in the pooled population (FIG. 7B). Hydroxydecanoic acid was significantly decreased at the 7 g dose in healthy adults (FIG. 8A) and at the 30 g dose in the ELD (FIG. 8B).

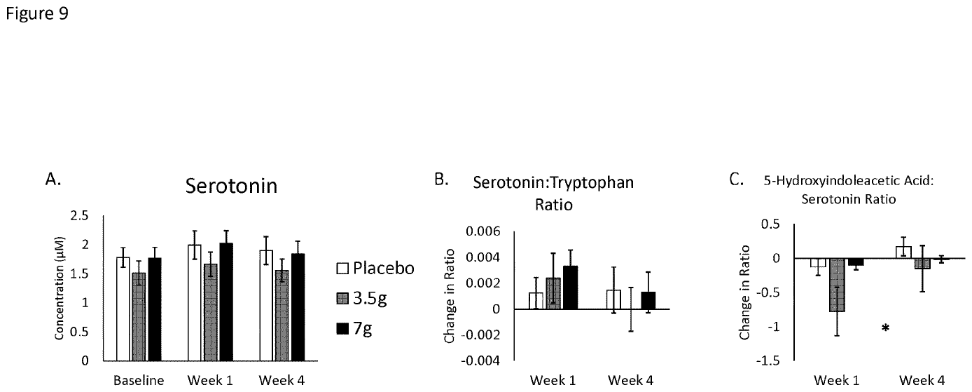

Serotonin levels were nominally higher in all three doses without significant differences (FIG. 9A), and while the ratio of serotonin to tryptophan was not significantly different (FIG. 9B), the ratio of 5-hydroxyindoleacetic acid to serotonin was significantly affected by treatment (FIG. 9C).

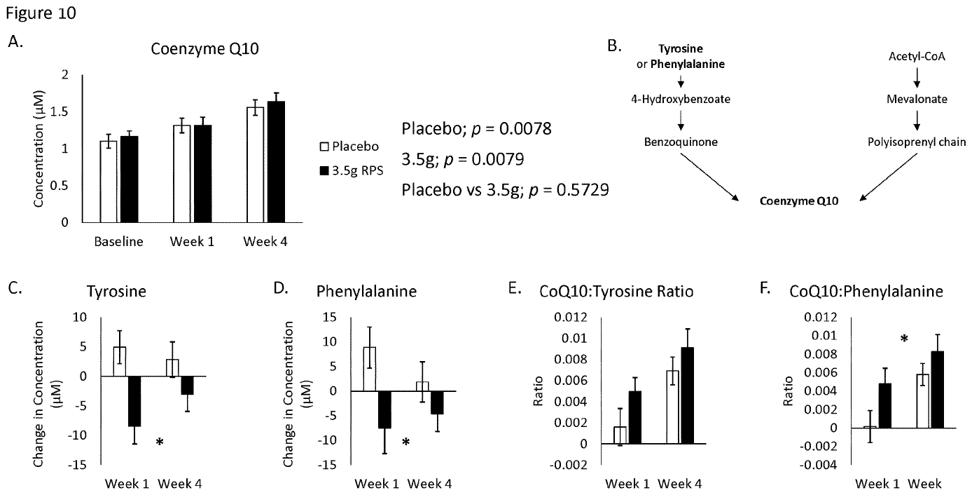

Coenzyme Q10 levels significantly increased in response to both the 3.5 g dose and placebo (FIG. 10A). Coenzyme Q10 is synthesized from several other molecules including amino acids tyrosine and phenylalanine (FIG. 10B). Tyrosine levels (FIG. 10C) and phenylalanine levels (FIG. 10D) were significantly decreased in the 3.5 g RPS arms. While the ratio of coenzyme Q10 to tyrosine trended towards significance (FIG. 10E), the ratio of Coenzyme Q10 to phenylalanine was significantly greater in participants receiving the 3.5 g dose (FIG. 10F).

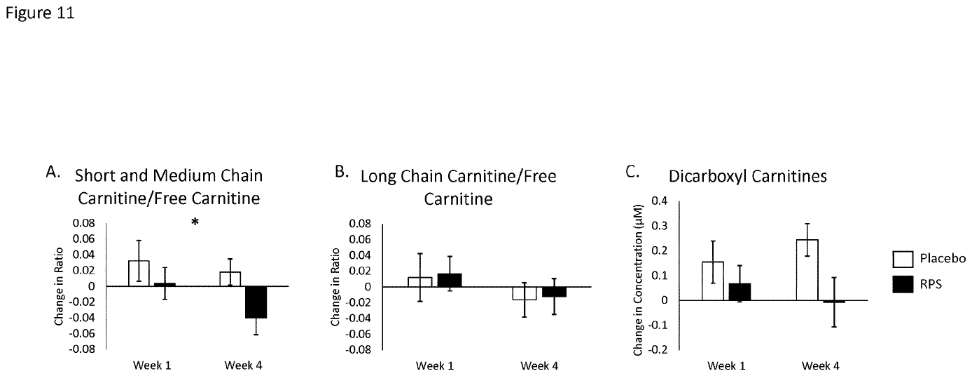

The ratio of short-and medium-chain carnitines to free carnitines was significantly reduced in participants receiving the 3.5 g dose compared to placebo (FIG. 11A), while the ratio of long-chain carnitines to free carnitines (FIG. 11B) and the levels of dicarboxyl carnitines (FIG. 11C) were not affected by treatment.

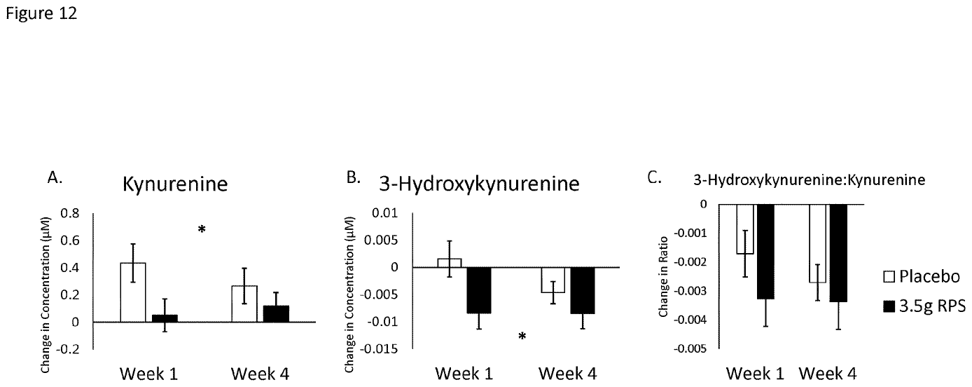

Finally, participants receiving the placebo experienced an increase in kynurenine (FIG. 12A) while those consuming 3.5 g RPS per day dose experienced a decrease in levels of 3-hydroxykynurenine (FIG. 12B). The ratio of 3-hydroxykynurenine to kynurenine was not affected by treatment (FIG. 12C). Pregnenolone sulfate and 3-beta-hydroxy-5-cholestenoic acid are both cholesterol-derived metabolites that have influences on neuronal function [10] [11]. Pregnenolone sulfate is a cognitive enhancer and modulator of neurotransmission, playing a role in learning and memory [11]. Serum pregnenolone sulfate levels are significantly lower in women with menstrual-related migraine (mean 39.58 ng/ml) compared to controls (mean 55.82 ng/ml) [12] and baseline serum pregnenolone sulfate levels were inversely correlated with MATRICS Consensus Cognitive Battery scores in schizophrenia patients treated with pregnenolone [13], indicating that increasing serum levels of pregnenolone sulfate has therapeutic benefits. 3-beta-hydroxy-5-cholestenoic acid is normally found in blood at 67.2 +/−27.9 ng/ml (mean +/−SD) [14] and levels are reduced in patients with ileal resection [15]. 3-beta-hydroxy-5-cholestenoic acid is a selective gamma secretase modulator with specific action on amyloid beta 42 (AB42), decreasing the Aβ42-to-Aβ38 ratio suggesting that increasing 3-beta-hydroxy-5-cholestenoic acid could be beneficial in mitigating Alzheimer's disease [10]. Resistant starch has not previously been linked to changes in either of these metabolites. Bile acids are produced by the liver from cholesterol and released from the gall bladder to emulsify dietary fats and promote the absorption of lipids and lipid-soluble nutrients. Resistant corn starch decreased excretion of bile acid in feces [16] [17], reduced total and secondary bile acids [18], had no effect on bile acids [19] [20], and increased bile acid excretion [21] [22] [23]. RS from adzuki and tebou beans, as well as from potato, enhanced fecal bile acid excretion [24] [25] [26] [27]. RS has previously been shown to have differing effects on taurochenodeoxycholate [22]

, and RS from lotus seed has been shown to convert taurocholic acid to taurodeoxycholic acid but not to influence taurine conjugation per se [29]. Taken together, our findings are the first to show that RPS consistently reduce serosal abundance of taurine and glycine conjugated bile acids, potentially by enhancing bile salt hydrolase activity of gut microbiota, and decrease abundance of muricholic acid and ursodeoxycholic acid 3-glucuronide.

Abundance of 5-hydroxylysine and 4-hydroxyproline in serum and urine are markers of collagen breakdown, and increased levels have been characterized in several disease contexts, including diabetic nephropathy [30], viral hepatitis [31], fibrodysplasia ossificans progressive [32], Paget's disease [33], chronic bronchitis, pneumonia, and emphysema [34], decompensated cirrhosis [35], mucopolysaccharidoses and irritable bowel syndrome [36] [37]. Serum levels of this metabolite rise following exercise [38] [39] [40], in which collagen in skeletal muscle is degraded during the rebuilding of muscle fibers. RPS-dependent inhibition of collagen degradation could be due to reduced inflammation via lower histamine levels, as collagenase activity is closely tied to neutrophil migration in response to histamine [41], or due to improved mitochondrial function and reduced production of reactive oxygen species, which are known to increase collagenase activity [42]. To our knowledge, this is the first report linking RPS consumption and decreases in serum 5-hydroxylysine or 4-hydroxyproline, or the promotion of collagen integrity.

The amine histamine is released at local sites of inflammation by mast cells, where it acts as a vasodilator to facilitate an enhanced immune response [43]. Histamine levels are elevated in individuals with chronic urticaria [44], histamine intolerance, atopic eczema [45], food allergies, major depressive disorder [46], and idiopathic anaphylaxis [47]. Normal basal plasma histamine levels range from 2.7-9.0 nM and symptoms of HIT, including elevated gastric acid secretion and increased heart rate, are reported in people with levels above 9 nM [48]. Histamine also plays a role as a neurotransmitter and RS supplementation of rats fed a high-fat diet reverse histamine receptor insensitivity caused by the high fat diet [49]. However, a role for RS in modulating histamine levels has not been previously described. Putrescine and spermidine are both polyamines that are formed in subsequent steps following ornithine decarboxylation. Polyamines play important roles in cellular growth, as indicated by elevated polyamine levels in proliferating cancer cells [50]. Polyamine levels are negatively regulated via acetylation and are normally found in serum from healthy individuals [51], and enhancing the enzymes responsible for polyamine acetylation is a pharmacological strategy that has been used in chemotherapeutic development [52]. Notably, higher N-acetyl spermidine levels are associated with poor outcomes in people with COVID-19 [53] [54] [55] and high levels of this metabolite are associated with cardiovascular disease [56], as well as various cancers, including hepatocellular carcinoma [57], colon cancer [58], astrocytoma [59], and breast cancer [60]. To our knowledge, we are the first to describe a relationship between RS consumption and reductions in N-acetyl putrescine or reductions in N-acetyl spermidine. Acylcarnitines are carnitines esterified to fatty acids via acyl-coenzyme A and have historically been measured in cases of inborn errors of fatty acid oxidation, but short chain acylcarnitines have recently been approved by the Food and Drug Administration for dietary supplement purposes [61]. Endogenous propionyl carnitine levels significantly increase in response to pioglitazone in obese women with polycystic ovarian syndrome, which is associated with improved fatty acid metabolism and improved mitochondrial function [62]. Propionyl carnitine supplementation promotes cardiac and vascular health, mitochondrial function, lipid peroxidation, and erectile function [63] [64] [65] [66] [67] [68] [69]. There is also evidence supporting a role for propionyl carnitine in exercise performance [65] [70]. While resistant starch chemically modified via propionate conjugation (RS type 4) has been shown to elevate propionyl carnitine in cats, these effects were due to the propionyl moiety, as acetylated resistant starch did not elevate propionyl carnitine [71]. Our data are the first to demonstrate that native RPS (RS type 2) significantly elevates propionyl carnitine and heptanoyl carnitine.

Serotonin is a monoaminergic neurotransmitter derived from the essential amino acid tryptophan and it plays important roles in multiple physiological aspects, including anxiety and the stress response [72]. Serotonin levels can be decreased by dietary tryptophan depletion and maintaining adequate serotonin levels continues to be a major therapeutic avenue for individuals with clinical depression [73]. Serotonin is degraded via monoamine oxidase (MAO) into 5-hydroxyindoleacetic acid [74] and MAO inhibitors are pharmacological agents used to treat neurological conditions like depression by increasing serotonin levels [75]. Our data are the first to demonstrate that native RPS (RS type 2) significantly decreases the ratio of 5-hydroxyindoleacetic acid to serotonin, potentially through microbially-derived metabolites originating from RPS fermentation in the gut that influence the activity of MAO.

Coenzyme Q10 is an antioxidant that is endogenously synthesized in a complex manner from either tyrosine or phenylalanine, to produce 4-hydroxybenzoic acid then benzoquinone, and acetyl COA to produce polyisoprenyl side chain via the mevalonate pathway, before condensation of benzoquinone and the polyisoprenyl side chain [76]. Coenzyme Q10 functions as an electron carrier within the mitochondrion during oxidative phosphorylation and plays an important role as a lipid-soluble antioxidant, protecting both mitochondrial and other cell membranes from free radical-induced oxidative stress [76]. Tissue levels of coenzyme Q10 correlate with oxidative function, with levels highest in heart, kidney, liver, and muscle, further supporting the physiological protective status of this substance [77]. RPS administration led to a 0.4 mg/mL increase in serum coenzyme Q10 levels, corresponding to 40% greater levels after 4 weeks. Coenzyme Q10 levels significantly increased in both RPS and placebo groups over time, but the ratio of coenzyme Q10 to phenylalanine was significantly greater in the RPS arm, indicating the RPS enhances coenzyme Q10 synthesis. Given that RPS administration also enhances β-oxidation, it is possible that enhanced coenzyme Q10 synthesis reflects overall improvements in mitochondrial function in response to RPS supplementation.

In addition to supplementation with carnitines to support mitochondrial function, the ratios of acyl carnitines to free carnitines can be informative, depending on their length. Short-and medium-chain carnitine to free carnitine ratios tend to accumulate in cases of mitochondrial dysfunction, where failure to catabolize the acyl chains via β-oxidation leads to a relative accumulation of shorter acyl carnitines [78]. Surprisingly, we see a reduction in the ratio of short-and medium-chain carnitines to free carnitine, further demonstrating that RPS enhances mitochondrial function and -oxidation. This shift in ratios could reflect enhanced mitochondrial utilization of microbially-derived metabolites generated by RPS fermentation in the gut.

While tryptophan is the precursor for serotonin production, most tryptophan is metabolized into kynurenine [79]. Kynurenine is further metabolized to 3-hydroxykynurenine, a substance with cytotoxic and neurotoxic activities attributed to reactive oxygen species generation [80]. Levels of 3-hydroxykynurenine are elevated in patients with Alzheimer's disease [81] and mouse models of Huntington disease [82], and interventions that decrease 3-hydroxykynurenine levels are being sought [83]. To our knowledge, there are no known links between resistant starch supplementation and reductions in 3-hydroxykynurenine. Decreases in 3-hydroxykynurenine could be secondary to RPS-dependent decreases in 5-hydroxyindoleacetic acid or could act in parallel, reflecting gut-derived influence on serotonin metabolism.

Hydroxydecanoic acid collectively refers to all forms of decanoic acid that have been hydroxylated at various carbon positions, including those that are present in royal jelly and those that are used to artificially modify mitochondrial potassium ATP channels [84] [85]. While the role of hydroxydecanoic acid in health is not well defined, our data are the first to demonstrate that RPS supplementation significantly reduces the abundance of this metabolite. As discussed herein and as summarized above, RPS supplementation significantly altered the metabolomic profile of several metabolites associated with improved health. However, changes in short chain fatty acids typically associated with RPS supplementation, like butyrate, were not detected. RS was an historically abundant and important form of dietary fiber in human diets [2], and it is likely that human physiology evolved to rely on dietary sources of RS to provide adequate nutrition and positively regulate levels of biologically relevant metabolites.

While not wishing to be bound to a particular theory or hypothesis, supplementation with RPS, an indigestible form of dietary fiber, may influence the activity of digestive enzymes and/or alter the absorption of nutrients in the intestines, producing changes in the host metabolome. It is also possible that providing RS to the gut microbiota produces changes in bacterial-derived metabolites and that the changes in host metabolomics are either directly due to changes in absorption of these metabolites from the gut microbiota or indirectly from host responses to microbiota-produced metabolites. That is, these host metabolites are microbially-influenced in that changes to the gut microbiota has an effect on the levels of these host metabolites, as discussed herein.

In summary, inconsistent with what is predicted by the literature, we failed to detect significant changes in SCFAs in serum, including a lack of change in the RPS fermentation end product butyrate. Instead, we find that RPS supplementation, across a range of doses and durations, positively influences the abundance of numerous metabolites not previously associated with RS fermentation by the gut microbiota. Further research is needed to elucidate the mechanisms by which the above-described metabolite changes can be attributed to the consumption of RPS.

According to an aspect of the invention, there is provided a method of modifying levels of a microbially-influenced metabolite in human serum generated by resistant potato starch fermentation in the human's gut comprising administering to said human an effective amount of a suitable resistant potato starch on a dosage regimen.

In some embodiments, the microbially-influenced metabolite is collagen, a collagen breakdown product, a small organic acid, a medium chain fatty acid, a small chain fatty acid, an amine, an amino acid, a carnitine, or a bile acid.

In some embodiments, the microbially-influenced metabolite is selected from the group consisting of collagen, 5-hydroxylysine, 4-hydroxyproline, pregnenolone sulfate, 3-beta-hydroxy-5-cholestenoic acid, 3-hydroxykynurenine, histamine, N-acetyl putrescine, N-acetyl spermidine, acylcarnitine, carnitine, propionyl carnitine, serotonin, coenzyme Q10 and hydroxydecanoic acid.

According to an aspect of the invention, there is provided a method of increasing circulating neurosteroid pregnenolone sulfate levels in an individual in need of such treatment comprising administering to said individual an effective amount of a suitable resistant potato starch on a dosage regimen.

As such, in this context, “an individual in need of such treatment” may be an individual whose circulating serum pregnenolone sulfate levels are lower than a control individual, for example, lower than 56 ng/ml or lower than 40 ng/ml or an individual who suffers from frequent migraines, for example, menstrual-related migraines or headaches or a neuro-cognitive disorder, for example, has a cognitive deficit, schizophrenia or some other neurological condition or is a student, is study or otherwise is in need of enhanced memory function.

According to another aspect of the invention, there is provided use of native RPS (RS type 2) for increasing circulating serum pregnenolone sulfate levels or for reducing the severity and/or frequency of migraines compared to an individual of similar age and condition not administered the dosage regimen (referred to herein elsewhere as a “control individual”).

According to another aspect of the invention, there is provided use of native RPS (RS type 2) for treating frequent migraines, for example, menstrual-related migraines or headaches.

According to another aspect of the invention, there is provided use of native RPS (RS type 2) for treating a neuro-cognitive disorder, for example, a cognitive deficit, schizophrenia, or some other neurological condition.

According to another aspect of the invention, wherein the individual is studying, or is a student, there is provided use of native RPS (RS type 2) for enhanced memory function.

In another aspect of the invention, there is provided a method of increasing circulating levels of 3-beta-hydroxy-5-cholestenoic acid in an individual in need of such treatment comprising administering to said individual an effective amount of a suitable resistant potato starch on a dosage regimen.

As such, in this context, “an individual in need of such treatment” may be an individual whose circulating 3-beta-hydroxy-5-cholestenoic acid is lower than a control individual, for example, is below 67.2 ng/ml or below 67 ng/ml or who has or is suffering from Alzheimer's disease or has a hereditary risk of developing Alzheimer's disease or requires a modulator of gamma secretase activity.

According to another aspect of the invention, there is provided use of native RPS (RS type 2) for increasing circulating 3-beta-hydroxy-5-cholestenoic acid.

According to another aspect of the invention, there is provided use of native RPS (RS type 2) for treating or reducing the severity of symptoms associated with Alzheimer's disease.

In another aspect of the invention, there is provided a method of decreasing circulating levels of glycine-conjugated bile acids and/or taurine conjugated bile acids in an individual in need of such treatment comprising administering to said individual an effective amount of a suitable resistant potato starch on a dosage regimen.

As such, in this context, “an individual in need of such treatment” may be an individual who has elevated circulating serosal levels of taurine-conjugated bile acids and glycine conjugated bile acids compared to a control individual.

According to another aspect of the invention, there is provided use of native RPS (RS type 2) to reduce circulating serosal levels of taurine-conjugated bile acids and glycine conjugated bile acids.

According to another aspect of the invention, there is provided use of native RPS (RS type 2) for treating dyslipidemia, high cholesterol, obesity, metabolic syndrome, diabetes, or a related metabolic disorder.

In another aspect of the invention, there is provided a method of enhancing probiotic activity in an individual in need of such treatment comprising administering to said individual an effective amount of a suitable resistant potato starch on a dosage regimen.

As such, in this context, “an individual in need of such treatment” may be an individual who is also taking probiotics, either in a formulation containing RPS or another format without RPS.

According to another aspect of the invention, there is provided use of native RPS (RS type 2) to support probiotic survival or viability.

According to another aspect of the invention, there is provided use of native RPS (RS type 2) for treating antibiotic associated diarrhea, idiopathic diarrhea, constipation, or other forms of digestive distress.

In another aspect of the invention, there is provided a method of decreasing circulating levels of bile acids in an individual in need of such treatment comprising administering to said individual an effective amount of a suitable resistant potato starch on a dosage regimen.

Alternatively, “an individual in need of such treatment” may be an individual who has elevated circulating levels of bile acids compared to a control individual and/or is an individual who has or is at risk of developing dyslipidemia, high cholesterol, obesity, metabolic syndrome, diabetes, or a related metabolic disorder.

According to another aspect of the invention, there is provided use of native RPS (RS type 2) to decrease circulating levels of bile acids.

According to another aspect of the invention, there is provided use of native RPS (RS type 2) for treating dyslipidemia, high cholesterol, obesity, metabolic syndrome, diabetes, or a related metabolic disorder.

In another aspect of the invention, there is provided a method of decreasing circulating levels of the tryptophan metabolite 3-hydroxykynurenine in an individual in need of such treatment comprising administering to said individual an effective amount of a suitable resistant potato starch on a dosage regimen.

As such, in this context, an “individual in need of such treatment” may be an individual who has elevated circulating levels of 3-hydroxykynurenine compared to a control individual, for example, baseline urine 3-hydroxykynurenine levels greater than 450 μg/g creatinine, or an individual suffering from or predisposed to Alzheimer's disease, Huntington disease, Parkinson's disease, vitamin B6 deficiency, or is receiving hydrocortisone treatment or who is pregnant.

In another aspect of the invention, there is provided use of native RPS (RS type 2) to decrease circulating levels of 3-hydroxykynurenine.

In another aspect of the invention, there is provided use of native RPS (RS type 2) for treating Alzheimer's disease, Huntington disease, Parkinson's disease, vitamin B6 deficiency, or in supporting individuals who are receiving hydrocortisone treatment or who are pregnant.

In another aspect of the invention, there is provided a method of decreasing circulating levels of the amine histamine in an individual in need of such treatment comprising administering to said individual an effective amount of a suitable resistant potato starch on a dosage regimen.

As such, in this context, an “individual in need of such treatment” may be an individual who has elevated circulating levels of histamine compared to a control individual, for example, baseline serum histamine levels greater than 10 nmol/L, or an individual suffering from or prone to enhanced immune responses, chronic urticaria, histamine intolerance, atopic eczema, food allergies, major depressive disorder, or idiopathic anaphylaxis.

In another aspect of the invention, there is provided use of native RPS (RS type 2) to decrease circulating levels of histidine.

In another aspect of the invention, there is provided use of native RPS (RS type 2) for treating an enhanced immune response, inflammation, itching, swelling, an allergic reaction, chronic urticaria, histamine intolerance, mast cell activation syndrome, atopic eczema, allergies, food allergies, major depressive disorder, or idiopathic anaphylaxis.

In another aspect of the invention, there is provided a method of decreasing circulating levels of the collagen breakdown product 5-hydroxylysine in an individual in need of such treatment comprising administering to said individual an effective amount of a suitable resistant potato starch on a dosage regimen.

As such, in this context, an “individual in need of such treatment” may be an individual who has elevated circulating levels of 5-hydroxylysine compared to a control individual, for example, baseline serum hydroxylysine levels greater than 0.24 μmol/L. Such an individual may suffer from diabetic nephropathy, viral hepatitis, fibrodysplasia ossificans progressive, Paget's disease, chronic bronchitis, pneumonia, emphysema, decompensated cirrhosis, mucopolysaccharidoses, irritable bowel syndrome, impaired collagen metabolism, has a hereditary condition that affects collagen synthesis, is elderly, or otherwise subjects their skin to environmental exposures that promote collagen degradation.

In another aspect of the invention, there is provided use of native RPS (RS type 2) to decrease circulating levels of 5-hydroxylysine.

In another aspect of the invention, there is provided use of native RPS (RS type 2) for treating diabetic nephropathy, viral hepatitis, fibrodysplasia ossificans progressive, Paget's disease, chronic bronchitis, pneumonia, emphysema, decompensated cirrhosis, mucopolysaccharidoses, irritable bowel syndrome, impaired collagen metabolism, or a hereditary condition that affects collagen synthesis.

In another aspect of the invention, there is provided a method of decreasing circulating levels of the collagen breakdown product 4-hydroxyproline in an individual in need of such treatment comprising administering to said individual an effective amount of a suitable resistant potato starch on a dosage regimen.

As such, in this context, an “individual in need of such treatment” may be an individual who has elevated circulating levels of 5-hydroxylysine or 4-hydroxyproline compared to a control individual, for example, baseline serum hydroxylysine levels greater than 13 μmol/L. Such an individual may suffer from McCune-Albright syndrome, Paget's disease, scleroderma, impaired collagen metabolism, has a hereditary condition that affects collagen synthesis, is elderly, or otherwise subjects their skin to environmental exposures that promote collagen degradation.

In another aspect of the invention, there is provided use of native RPS (RS type 2) to decrease circulating levels of 4-hydroxyproline.

In another aspect of the invention, there is provided use of native RPS (RS type 2) for treating McCune-Albright syndrome, Paget's disease, scleroderma, impaired collagen metabolism, or a hereditary condition that affects collagen synthesis.

In another aspect of the invention, there is provided use of native RPS (RS type 2) to prevent collagen degradation in an individual in need of such treatment comprising administering to said individual an effective amount of a suitable resistant potato starch on a dosage regimen.

As such, in this context, an “individual in need of such treatment” may be an individual who has elevated circulating levels of 5-hydroxylysine or 4-hydroxyproline compared to a control individual, for example, baseline serum hydroxylysine levels greater than 13 μmol/L. Such an individual may suffer from McCune-Albright syndrome, Paget's disease, scleroderma, impaired collagen metabolism, has a hereditary condition that affects collagen synthesis, is elderly, or otherwise subjects their skin to environmental exposures that promote collagen degradation.

In another aspect of the invention, there is provided a method of decreasing circulating levels of the polyamine N-acetyl putrescine in an individual in need of such treatment comprising administering to said individual an effective amount of a suitable resistant potato starch on a dosage regimen.

As such, in this context, an “individual in need of such treatment” may be an individual having elevated levels of N-acetyl putrescine compared to a control individual.

In another aspect of the invention, there is provided use of native RPS (RS type 2) for reducing levels of N-acetyl putrescine. In another aspect of the invention, there is provided a method of decreasing circulating levels of the polyamine N-acetyl spermidine in an individual in need of such treatment comprising administering to said individual an effective amount of a suitable resistant potato starch on a dosage regimen.

As such, in this context, an “individual in need of such treatment” may be an individual having elevated levels of N-acetyl spermidine compared to a control individual, for example, baseline serum N-acetyl spermidine levels greater than 0.5 nmol/L, or an individual infected or suspected of having been infected with coronavirus, or who has or is at genetic risk of developing cancer or who has or is at genetic risk of developing cardiovascular disease.

In another aspect of the invention, there is provided use of native RPS (RS type 2) for reducing levels of N-acetyl spermidine.

In another aspect of the invention, there is provided use of native RPS (RS type 2) for treating a coronavirus infection, for example, lessening or reducing the severity of one or more symptoms related to a coronavirus infection, for example, difficulty breathing, clotting, and requiring breathing assistance. In another aspect of the invention, there is provided a method of increasing circulating levels of the polyamines in an individual in need of such treatment comprising administering to said individual an effective amount of a suitable resistant potato starch on a dosage regimen.

In another aspect of the invention, there is provided a method of increasing circulating acylcarnitine levels in an individual in need of such treatment comprising administering to said individual an effective amount of a suitable resistant potato starch on a dosage regimen.

As such, in this context, an “individual in need of such treatment” is an individual with reduced circulating levels of acyl carnitine compared to a control individual.

According to another aspect of the invention, there is provided use of native RPS (RS type 2) to elevate circulating levels of acyl carnitine. In another aspect of the invention, there is provided a method of decreasing the serum ratio of small-and medium-chain carnitines to free carnitine in an individual in need of such treatment comprising administering to said individual an effective amount of a suitable resistant potato starch on a dosage regimen.

As such, in this context, an “individual in need of such treatment” may be an individual having an elevated ratio of small-and medium-chain carnitines to free carnitine compared to a control individual, an individual with impaired mitochondrial function, or who has intestinal permeability, due to non-steroidal anti-inflammatory drug (NSAID) use, chemotherapy, viral infection, such as human immunodeficiency virus (HIV), as a co-morbidity to irritable bowel disease or inflammatory bowel disease (e.g. Crohn's disease or colitis), diabetes, or as a primary affliction.

In another aspect of the invention, there is provided use of native RPS (RS type 2) for reducing the ratio of small-and medium-chain carnitines to free carnitine.

In another aspect of the invention, there is provided use of native RPS (RS type 2) for treating mitochondrial dysfunction, for example, decreasing reactive oxygen species (e.g., Free radical) production.

In another aspect of the invention, there is provided a method of increasing circulating levels of propionyl carnitine in an individual in need of such treatment comprising administering to said individual an effective amount of a suitable resistant potato starch on a dosage regimen.

As such, in this context, an “individual in need of such treatment” is an individual with reduced circulating levels of propionyl carnitine compared to a control individual, for example, has baseline serum propionyl carnitine levels <0.39 μmol/L or is an athlete, exercising, or otherwise undergoing training to improve their muscle strength and/or body coordination or a male in need of improved sexual performance, improved erectile function, or circulation enhancement.

According to another aspect of the invention, there is provided use of native RPS (RS type 2) to elevate circulating levels of acyl carnitine.

According to another aspect of the invention, there is provided use of native RPS (RS type 2) for improving sexual performance, improving erectile function, or circulation enhancement.

In another aspect of the invention, there is provided a method of increasing circulating levels of serotonin in an individual in need of such treatment comprising administering to said individual an effective amount of a suitable resistant potato starch on a dosage regimen.

As such, in this context, an “individual in need of such treatment” is an individual with reduced circulating levels of serotonin compared to a control individual, for example, has baseline serum serotonin levels <50 ng/mL, who is experiencing anxiety, or neurological conditions, such as clinical depression.

According to another aspect of the invention, there is provided use of native RPS (RS type 2) to elevate circulating levels of serotonin.

According to another aspect of the invention, there is provided use of native RPS (RS type 2) for inhibiting monoamine oxidase (MAO) activity.

In another aspect of the invention, there is provided a method of increasing circulating levels of coenzyme Q10 in an individual in need of such treatment comprising administering to said individual an effective amount of a suitable resistant potato starch on a dosage regimen.

As such, in this context, an “individual in need of such treatment” is an individual with reduced circulating levels of coenzyme Q10 compared to a control individual, for example, has baseline serum coenzyme Q10 levels <0.5 μmol/L, is an athlete, or is suffering from heart conditions, such as migraines, congestive heart failure, high blood pressure, or high cholesterol, has or is suspected of having diabetes or Parkinson's disease, or is taking statin drugs and has statin-induced myopathy.

According to another aspect of the invention, there is provided use of native RPS (RS type 2) to elevate circulating levels of coenzyme Q10.

According to another aspect of the invention, there is provided use of native RPS (RS type 2) for improving mitochondrial function, reducing free radical effects, and/or reducing the effects of oxidative stress.

According to another aspect of the invention, there is provided a method of decreasing circulating levels of hydroxydecanoic acid in an individual in need of such treatment comprising administering to said individual an effective amount of a suitable resistant potato starch on a dosage regimen.

As such, in this context, an “individual in need of such treatment” is an individual with elevated circulating levels of hydroxydecanoic acid compared to a control individual.

Accordingly, in another aspect of the invention, there is provided use of native RPS (RS type 2) to reduce the abundance of hydroxydecanoic acid.

In some embodiments of the invention, the resistant potato starch is administered daily for a period of 1-12 weeks. In this context, “daily” does not necessarily mean “every day” but may mean for example 19 out of 20 days, 9 out of 10 days, 17 out of 20 days or 8 out of 10 days.

As discussed herein, the effective amount may be, for example, 0.5 to 40 g, or 0.5 to 30 g, or 0.5 to 7 g, or 0.5 to 3.5 g of resistant starch.

While the preferred embodiments of the invention have been described above, it will be recognized and understood that various modifications may be made therein, and the appended claims are intended to cover all such modifications which may fall within the spirit and scope of the invention.

### REFERENCES

[1] H. Englyst, S. Kingman, G. Hudson and J. Cummings, “Measurement of resistant starch in vitro and in vivo,” Br J Nutr, vol. 75, no. 5, pp. 749-755, 1996.
[2] D. Miketinas, K. Shankar, M. Maiya and M. Patterson, “Usual Dietary Intake of Resistant Starch in US Adults from NHANES 2015-2016,” J Nutr, vol. 150, no. 10, pp. 2738-2747, 2020.
[3] S. Landon, C. Colyer and H. Salman, “The resistant starch report,” Food Australia Supplement, 2012.
[4] R. Beger, W. Dunn, M. Schmidt, S. Gross, J. Kirwan, M. Cascante, L. Brennan, D. Wishart, M. Oresic, T. Hankemeier, D. Broadhurst, A. Lane, K. Suhre, G. Kastenmüller, S. Sumner, I. Thiele, O. Fiehn, R. Kaddurah-Daouk and e. al, “Metabolomics enables precision medicine: ”A White Paper, Community Perspective“,” Metabolomics, vol. 12, no. 10, p. 149, 2016.
[5] J. Teichmann and D. Cockburn, “In vitro Fermentation Reveals Changes in Butyrate Production Dependent on Resistant Starch Source and Microbiome Composition,” Front Microbiol, vol. 12, p. 640253, 2021.
[6] M. Alfa, D. Strang, P. Tappia, M. Graham, G. Van Domselaar, J. Forbes, V. Laminman, N. Olson, P. DeGagne, D. Bray, B.-L. Murray, B. Dufault and L. Lix, “A randomized trial to determine the impact of a digestion resistant starch composition on the gut microbiome in older and mid-age adults,” Clin Nutr, vol. 37, no. 3, pp. 797-807, 2018.
[7] A. Bird, M. Conlon, C. Christophersen and D. Topping, “Resistant starch, large bowel fermentation and a broader perspective of prebiotics and probiotics,” Benef Microbes, vol. 1, no. 4, pp. 423-431, 2010.
[8] S. Bridgeman, W. Northrop, P. Melton, G. Ellison, P. Newsholme and C. Mamotte, “Butyrate generated by gut microbiota and its therapeutic role in metabolic syndrome,” Pharmacol Res, vol. 160, p. 105174, 2020.
[9] M. Alfa, D. Strang, P. Tappia, N. Olson, P. DeGagne, D. Bray, B.-L. Murray and B. Hiebert, “A Randomized Placebo Controlled Clinical Trial to Determine the Impact of Digestion Resistant Starch MSPrebiotic on Glucose, Insulin, and Insulin Resistance in Elderly and Mid-Age Adults,” Fronteirs in Medicine (Lausanne), vol. 4, p. 260, 2018.
[10] J. Jung, A. Price, T. Ladd, Y. Ran, H. Park, C. Ceballos-Diaz, L. Smithson, G. Hochhaus, Y. Tang, R. Akula, S. Ba, E. Koo, G. Shapiro, K. Felsenstein and T. Golde, “Cholestenoic acid, an endogenous cholesterol metabolite, is a potent y-secretase modulator,” Mol Neurodegener, vol. 10, p. 29, 2015.
[11] C. Smith, T. Gibbs and D. Farb, “Pregnenolone sulfate as a modulator of synaptic plasticity,” Psychopharmacology (Berl), vol. 231, no. 17, pp. 3537-3556, 2014.
[12] C. Rustichelli, E. Bellei, S. Bergamini, E. Monari, F. Lo Castro, C. Baraldi, A. Tomasi and A. Ferrari, “Comparison of pregnenolone sulfate, pregnanolone and estradiol levels between patients with menstrually-related migraine and controls: an exploratory study,” J Headache Pain, vol. 22, no. 1, p. 13, 2021.
[13] C. Marx, R. Keefe, R. Buchanan, R. Hamer, J. Kilts, D. Bradford, J. Strauss, J. Naylor, V. Payne, J. Lieberman, A. Savitz, L. Leimone, L. Dunn, P. Porcu, A. Morrow and L. Shampine, “Proof-of-concept trial with the neurosteroid pregnenolone targeting cognitive and negative symptoms in schizophrenia,” Neuropsychopharmacology, vol. 34, no. 8, pp. 1885-1903, 2009.
[14] M. Axelson, B. Mörk and J. Sjövall, “Occurrence of 3 beta-hydroxy-5-cholestenoic acid, 3 beta, 7alpha-dihydroxy-5-cholestenoic acid, and 7 alpha-hydroxy-3-oxo-4-cholestenoic acid as normal constituents in human blood,” J Lipid Res, vol. 29, no. 5, pp. 629-641, 1988.
[15] M. Axelson, B. Mörk, A. Aly, G. Walldius and J. Sjövall, “Concentrations of cholestenoic acids in plasma from patients with reduced intestinal reabsorption of bile acids,” J Lipid Res, vol. 30, no. 12, pp. 1883-1887, 1989.
[16] I. van Munster, A. Tangerman and F. Nagengast, “Effect of resistant starch on colonic fermentation, bile acid metabolism, and mucosal proliferation,” Dig Dis Sci, vol. 39, no. 4, pp. 834-842, 1994.
[17] A. Langkilde, H. Ekwall, I. Björck, N. Asp and H. Andersson, “Retrograded high-amylose corn starch reduces cholic acid excretion from the small bowel in ileostomy subjects,” Eur J Clin Nutr, vol. 52, no. 11, pp. 790-795, 1998.
[18] S. Hylla, A. Gostner, G. Dusel, H. Anger, H. Bartram, S. Christl, H. Kasper and W. Scheppach, “Effects of resistant starch on the colon in healthy volunteers: possible implications for cancer prevention,” Am J Clin Nutr, vol. 67, no. 1, pp. 136-142, 1998.
[19] M. Heijnen, J. van Amelsvoort, P. Deurenberg and A. Beynen, “Limited effect of consumption of uncooked (RS2) or retrograded (RS3) resistant starch on putative risk factors for colon cancer in healthy men,” Am J Clin Nutr, vol. 67, no. 2, pp. 322-331, 1998.
[20] M. Heijnen, J. van Amelsvoort, P. Deurenberg and A. Beynen, “Neither raw nor retrograded resistant starch lowers fasting serum cholesterol concentrations in healthy normolipidemic subjects,” Am J Clin Nutr, vol. 64, no. 3, pp. 312-318, 1996.
[21] K. Vanhoof and R. De Schrijver, “The influence of enzyme-resistant starch on cholesterol metabolism in rats fed on a conventional diet,” Br J Nutr, vol. 80, no. 2, pp. 193-198, 1998.
[22] E. Trautwein, K. Forgbert, D. Rieckhoff and H. Erbersdobler, “Impact of beta-cyclodextrin and resistant starch on bile acid metabolism and fecal steroid excretion in regard to their hypolipidemic action in hamsters,” Biochim Biophys Acta, vol. 1437, no. 1, pp. 1-12, 1999.
[23] M. Grubben, C. van den Braak, M. Essenberg, M. Olthof, A. Tangerman, M. Katan and F. Nagengast, “Effect of resistant starch on potential biomarkers for colonic cancer risk in patients with colonic adenomas: a controlled trial,” Dig Dis Sci, vol. 46, no. 4, pp. 750-756, 2001.
[24] H. Younes, M. Levrat, C. Demigne and C. Remesy, “Resistant starch is more effective than cholestyramine as a lipid-lowering agent in the rat,” Lipids, vol. 30, no. 9, pp. 847-853, 1995.
[25] K. Han, M. Fukushima, T. Kato, M. Kojima, K. Ohba, K. Shimada, M. Sekikawa and M. Nakano, “Enzyme-resistant fractions of beans lowered serum cholesterol and increased sterol excretions and hepatic mRNA levels in rats,” Lipids, vol. 38, no. 9, pp. 919-924, 2003.
[26] N. Hashimoto, Y. Ito, K. Han, K. Shimada, M. Sekikawa, D. Topping, A. Bird, T. Noda, H. Chiji and M. Fukushima, “Potato pulps lowered the serum cholesterol and triglyceride levels in rats,” J Nutr Sci Vitaminol (Tokyo), vol. 52, no. 6, pp. 445-450, 2006.
[27] T. Kanazawa, M. Atsumi, H. Mineo, M. Fukushima, N. Nishimura, T. Noda and H. Chiji, “Ingestion of gelatinized potato starch containing a high level of phosphorus decreases serum and liver lipids in rats,” J Oleo Sci, vol. 57, no. 6, pp. 335-343, 2008.
[28] Y. Koay, J. Wali, A. Luk, L. Macia, V. Cogger, T. Pulpitel, D. Wahl, S. Solon-Biet, A. Holmes, S. Simpson and J. O'Sullivan, “Ingestion of resistant starch by mice markedly increases microbiome-derived metabolites,” FASEB J, vol. 33, no. 7, pp. 8033-8042, 2019.
[29] S. Lei, L. Liu, L. Ding, Y. Zhang and H. Zeng, “Lotus seed resistant starch affects the conversion of sodium taurocholate by regulating the intestinal microbiota,” Int J Biol Macromol, vol. 186, pp. 227-236, 2021.
[30] N. Westberg, “Biochemical alterations of the human glomerular basement membrane in diabetes,” Diabetes, vol. 25, no. 2 SUPPL, pp. 920-924, 1976.
[31] M. Drozdz and E. Kucharz, “Serum hydroxyproline and hydroxylysine levels in viral hepatitis,” Med Interne, vol. 16, no. 1, pp. 79-82, 1978.
[32] N. Blumenkrantz and G. Asboe-Hansen, “Fibrodysplasia ossificans progressiva. Biochemical changes in blood serum, urine, skin, bone, and ectopic ossification,” Scand J Rheumatol, vol. 7, no. 2, pp. 85-89, 1978.
[33] R. Russell, D. Beard, E. Cameron, D. Douglas, A. Forrest, D. Guilland-Cumming, A. Paterson, J. Poser, C. Preston, A. Milford-Ward, S. Woodhead and J. Kanis, “Biochemical markers of bone turnover in Paget's disease,” Metab Bone Dis Relat Res, vol. 3, no. 4-5, pp. 255-262, 1981.
[34] E. Szoltysik and E. Kucharz, “Serum markers of collagen metabolism in patients with diseases of the respiratory system,” Rom J Intern Med, vol. 31, no. 2, pp. 119-122, 1993.
[35] E. Kucharz, “Serum hydroxyproline and hydroxylysine levels in patients with decompensated cirrhosis,” Rom K Intern Med, vol. 32, no. 4, pp. 271-274, 1994.
[36] N. Patel, P. Mills, J. Davison, M. Cleary, P. Gissen, B. Banushi, I. Doykov, M. Dorman, K. Mills and W. Heywood, “Free urinary glycosylated hydroxylysine as an indicator of altered collagen degradation in the mucopolysaccharidoses,” J Inherit Metab Dis, vol. 43, no. 2, pp. 309-317, 2020.
[37] M. Yamamoto, M. Pinto-Sanchez, P. Bercik and P. Britz-Mckibbin, “Metabolomics reveals elevated urinary excretion of collagen degradation and epithelial cell turnover products in irritable bowel syndrome patients,” Metabolomics, vol. 15, no. 6, p. 82, 2019.
[38] S. Brown, R. Child, S. Day and A. Donnelly, “Indices of skeletal muscle damage and connective tissue breakdown following eccentric muscle contractions,” Eur J Appl Physiol Occup Physiol, vol. 75, no. 4, pp. 369-374, 1997.
[39] T. Tofas, A. Jamurtas, I. Fatouros, M. Nikolaidis, Y. Koutedakis, E. Sinouris, N. Papageorgakopoulou and D. Theocharis, “Plyometric exercise increases serum indices of muscle damage and collagen breakdown,” J Strength Cond Res, vol. 22, no. 2, pp. 490-496, 2008.
[40] M. Behringer, J. Montag, Y. Kilian, P. Heaton and J. Mester, “Polyamines, myosin heavy chains, and collagen specific amino acids after a repeated bout of eccentric exercise,” Res Sports Med, vol. 24, no. 3, pp. 287-297, 2016.
[41] J. Labat-Robert, A. Fourtanier, B. Boyer-Lafargue and L. Robert, “Age dependent increase of elastase type protease activity in mouse skin. Effect of UV-irradiation,” J Photochem Photobiol B, vol. 57, no. 2-3, pp. 113-118, 2000.
[42] H. Saari, T. Sorsa, K. Suomalainen, S. Halinen and Y. Konttinen, “Reactive oxygen species as regulators of human neutrophil and fibroblast interstitial collagenases,” Int J Tissue React, vol. 14, no. 3, pp. 113-120, 1992.
[43] W. Dachman, G. Bedarida, T. Blaschke and B. Hoffman, “Histamine-induced venodilation in human beings involves both H 1 and H 2 receptor subtypes,” J Allergy Clin Immunol, vol. 93, no. 3, pp. 606-614, 1994.
[44] R. Asero and E. Madonini, “Bronchial hyperresponsiveness is a common feature in patients with chronic urticaria,” J Investig Allergol Clin Immunol, vol. 16, no. 1, pp. 19-23, 2006.
[45] L. Maintz, S. Benfadal, J. Allam, T. Hagemann, R. Fimmers and N. Novak, “Evidence for a reduced histamine degradation capacity in a subgroup of patients with atopic eczema,” J Allergy Clin Immunol, vol. 1117, no. 5, pp. 1106-1112, 2006.
[46] R. Tao, Z. Fu and L. Xiao, “Chronic Food Antigen-specific IgG-mediated Hypersensitivity Reaction as A Risk Factor for Adolescent Depressive Disorder,” Genomics Proteomics Bioinformatics, vol. 17, no. 2, pp. 183-189, 2019.
[47] M. Bilò, M. Martini, C. Tontini, O. Mohamed and M. Krishna, “Idiopathic anaphylaxis,” Clin Exp Allergy, vol. 49, no. 7, pp. 942-952, 2019.
[48] L. Maintz and N. Novak, “Histamine and histamine intolerance,” Am J Clin Nutr, vol. 85, no. 5, pp. 1185-1196, 2007.
[49] Z. Wu, Y. Yu, Y. Wu, C. Patch, A. Szabo and X.-F. Huang, “Reduction of histamine H1 receptor binding induced by high-fat diet can be prevented by DHA and dietary fiber in specific brain areas of male rats,” Brain Res Bull, vol. 97, pp. 119-125, 2013.
[50] R. Casero Jr, T. Murray Stewart and A. Pegg, “Polyamine metabolism and cancer: treatments, challenges and opportunities,” Nat Rev Cancer, vol. 18, no. 11, pp. 681-695, 2018.
[51] R. Smith, D. Bartos, F. Bartos, D. Grettie, W. Frick, R. Campbell and G. Daves Jr, “1-N-Acetylspermidine: occurrence in normal human serum,” Biomed Mass Spectrom, vol. 5, no. 9, pp. 515-517, 1978.
[52] T. Murray-Stewart, E. Ferrari, Y. Xie, F. Yu, L. Marton, D. Oupicky and R. Casero Jr, “Biochemical evaluation of the anticancer potential of the polyamine-based nanocarrier Nano11047,” PLoS ONE, vol. 12, no. 4, p. e 0175917, 2017.
[53] T. Thomas, D. Stefanoni, J. Reisz, T. Nemkov, L. Bertolone, R. Francis, K. Hudson, J. Zimring, K. Hansen, E. Hod, S. Spitalnik and A. D'Alessandro, “COVID-19 infection alters kynurenine and fatty acid metabolism, correlating with IL-6 levels and renal status,” JCI Insight, vol. 4, no. 14, p. e140327, 2020.
[54] M. Bourgin, L. Derosa, C. Silva, A. Goubet, A. Dubuisson, F. Danlos, C. Grajeda-Iglesias, L. Cerbone, A. Geraud, A. Laparra, F. Aprahamian, N. Nirmalathasan, F. Madeo, L. Zitvogel, G. Kroemer and S. Durand, “Circulating acetylated polyamines correlate with Covid-19 severity in cancer patients,” Aging (Albany NY), vol. 13, no. 17, pp. 20860-20885, 2021.
[55] F. Danlos, C. Grajeda-Iglesias, S. Durand, A. Sauvat, M. Roumier, D. Cantin, E. Colomba, J. Rohmer, F. Pommeret, G. Baciarello, C. Willekens, M. Vasse, F. Griscelli, J. Fahrner, A. Goubet, A. Dubuisson, L. Derosa, N. Nirmalathasan, D. Bredel and e. al, “Metabolomic analyses of COVID-19 patients unravel stage-dependent and prognostic biomarkers,” Cell Death Dis, vol. 12, no. 3, p. 258, 2021.
[56] S. Tabrez, M. Shait Mohammed, N. Jabir and M. Khan, “Identification of novel cardiovascular disease associated metabolites using untargeted metabolomics,” Bio/Chem, vol. 402, no. 6, pp. 749-757, 2021.
[57] M. Stepien, P. Keski-Rahkonen, A. Kiss, N. Robinot, T. Duarte-Salles, N. Murphy, G. Perlemuter, V. Viallon, A. Tjønneland, A. Rostgaard-Hansen, C. Dahm, K. Overvad, M. Boutron-Ruault, F. Mancini, Y. Mahamat-Saleh, K. Aleksandrova and e. al, “Metabolic perturbations prior to hepatocellular carcinoma diagnosis: Findings from a prospective observational cohort study,” Int J Cancer, vol. 148, no. 3, pp. 609-625, 2021.
[58] B. O'Brien, M. Hankewych, D. McCormick, R. Jacoby, T. Brasitus and A. Halline, “Urinary N1-acetylspermidine and N8-acetylspermidine excretion in normal humans and in patients with colorectal cancer,” Dig Dis Sci, vol. 40, no. 6, pp. 1269-1274, 1995.
[59] H. Kurihara, S. Matsuzaki, H. Yamazaki, T. Tsukahara and M. Tamura, “Relationship between tissue polyamine levels and malignancy in primary brain tumors,” Neurosurgery, vol. 32, no. 3, pp. 372-375, 1993.
[60] L. Persson and E. Rosengren, “Increased formation of N1-acetylspermidine in human breast cancer,” Cancer Lett, vol. 45, no. 2, pp. 83-86, 1989.
[61] M. Dambrova, M. Makrecka-Kuka, J. Kuka, R. Vilskersts, D. Nordberg, M. Attwood, S. Smesny, Z. Sen, A. Guo, E. Oler, S. Tian, J. Zheng, D. Wishart, E. Liepinsh and H. Schioth, “Acylcarnitines: Nomenclature, Biomarkers, Therapeutic Potential, Drug Targets, and Clinical Trials,” Pharmacol Rev, vol. 74, no. 3, pp. 506-551, 2022.
[62] N. Vigerust, P. Bohov, B. Bjørndal, R. Seifert, O. Nygård, A. Svardal, D. Glintborg, R. Berge and M. Gaster, “Free carnitine and acylcarnitines in obese patients with polycystic ovary syndrome and effects of pioglitazone treatment,” Fertil Steril, vol. 98, no. 6, pp. 1620-1626.e1, 2012.
[63] R. Micheletti, G. Giacalone, M. Canepari, S. Salardi, G. Bianchi and C. Reggiani, “Propionyl-L-carnitine prevents myocardial mechanical alterations due to pressure overload in rats,” Am J Physiol, vol. 266, no. 6 Pt 2, pp. H 2190-2197, 1994.
[64] T. Broderick, G. Haloftis and D. Paulson, “L-propionylcarnitine enhancement of substrate oxidation and mitochondrial respiration in the diabetic rat heart,” J Mol Cell Cardiol, vol. 28, no. 2, pp. 331-340, 1996.
[65] E. Brass and W. Hiatt, “The role of carnitine and carnitine supplementation during exercise in man and in individuals with special needs,” J Am Coll Nutr, vol. 17, no. 3, pp. 207-215, 1998.
[66] V. Gentile, P. Vicini, G. Prigiotti, A. Koverech and F. Di Silverio, “Preliminary observations on the use of propionyl-L-carnitine in combination with sildenafil in patients with erectile dysfunction and diabetes,” Curr Med Res Opin, vol. 20, no. 9, pp. 1377-1384, 2004.
[67] R. Bueno, M. Alvarez de Sotomayor, C. Perez-Guerrero, L. Gomez-Amores, C. Vazquez and M. Herrera, “L-carnitine and propionyl-L-carnitine improve endothelial dysfunction in spontaneously hypertensive rats: different participation of NO and COX-products,” Life Sci, vol. 77, no. 17, pp. 2082-2097, 2005.
[68] R. Bloomer, L. Tschume and W. Smith, “Glycine propionyl-L-carnitine modulates lipid peroxidation and nitric oxide in human subjects,” Int J Vitam Nutr Res, vol. 79, no. 3, pp. 131-141, 2009.
[69] D. Gianfrilli, R. Lauretta, C. Di Dato, C. Graziadio, C. Pozza, J. De Larichaudy, E. Giannetta, A. Isidori and A. Lenzi, “Propionyl-L-carnitine, L-arginine and niacin in sexual medicine: a nutraceutical approach to erectile dysfunction,” Andrologia, vol. 44, no. Suppl 1, pp. 600-604, 2012.
[70] G. Barker, S. Green, C. Askew, A. Green and P. Walker, “Effect of propionyl-L-carnitine on exercise performance in peripheral arterial disease,” Med Sci Sports Exerc, vol. 33, no. 9, pp. 1415-1422, 2001.
[71] K. Rochus, A. Cools, G. Janssens, L. Vanhaecke, B. Wuyts, T. Lockett, J. Clarke, V. Fievez and M. Hesta, “Dietary supplementation of propionylated starch to domestic cats provides propionic acid as gluconeogenic substrate potentially sparing the amino acid valine,” J Nutr Sci, vol. 3, p. e 16, 2014.
[72] E. Höglund, Ø. Øverli and S. Winberg, “Tryptophan Metabolic Pathways and Brain Serotonergic Activity: A Comparative Review,” Front Endocrinol, vol. 10, p. 00158, 2019.
[73] S. Jauhar, P. Cowen and M. Browning, “Fifty years on: Serotonin and depression,” J Psychopharmacol, vol. 37, no. 3, pp. 237-241, 2023.
[74] H. Jayamohananan, M. Manoj Kumar and T. Aneesh, “5-HIAA as a Potential Biological Marker for Neurological and Psychiatric Disorders,” Adv Pharm Bull, vol. 9, no. 3, pp. 374-381, 2019.
[75] R. Sub Laban and A. Saadabadi, “Monoamine Oxidase Inhibitors (MAOI),” 19 Jul. 2022. [Online]. Available: https://www.ncbi.nlm.nih.gov/books/NBK 539848/. [Accessed 30 5 2023].
[76] I. Hargreaves, R. Heaton and D. Mantle, “Disorders of Human Coenzyme Q10 Metabolism: An Overview,” Int J Mol Sci, vol. 21, no. 18, p. 6695, 2020.
[77] A. Martelli, L. Testai, A. Colletti and A. Cicero, “Coenzyme Q10: Clinical Applications in Cardiovascular Diseases,” Antioxidants (Basel), vol. 9, no. 4, p. 341, 2020.
[78] K. Crakes, C. Santos Rocha, I. Grishina, L. Hirao, E. Napoli, C. Gaulke, A. Fenton, S. Datta, J. Arredondo, M. Marco, S. Sankaran-Walters, G. Cortopassi, C. Giulivi and S. Dandekar, “PPARα-targeted mitochondrial bioenergetics mediate repair of intestinal barriers at the host-microbe intersection during SIV infection,” Proc Natl Acad Sci U S A, vol. 116, no. 49, pp. 24819-24829, 2019.
[79] S. Haq, J. Grondin and W. Khan, “Tryptophan-derived serotonin-kynurenine balance in immune activation and intestinal inflammation,” FASEB J, vol. 35, no. 10, p. e21888, 2021.
[80] S. Okuda, N. Nishiyama, H. Saito and H. Katsuki, “Hydrogen peroxide-mediated neuronal cell death induced by an endogenous neurotoxin, 3-hydroxykynurenine,” Proc Natl Acad Sci U S A, vol. 93, no. 22, pp. 12553-12558, 1996.
[81] M. Schwarz, G. Guillemin, S. Teipel, K. Buerger and H. Hampel, “Increased 3-Hydroxykynurenine serum concentrations differentiate Alzheimer's disease patients from controls,” Eur Arch Psychiatry Clin Neurosci, vol. 263, no. 4, pp. 345-352, 2013.
[82] P. Guidetti, G. Bates, R. Graham, M. Hayden, B. Leavitt, M. MacDonald, E. Slow, V. Wheeler, B. Woodman and R. Schwarcz, “Elevated brain 3-hydroxykynurenine and quinolinate levels in Huntington disease mice,” Neurobiol Dis, vol. 23, no. 1, pp. 190-197, 2006.
[83] T. Hughes, O. Guner, E. Iradukunda, R. Phillips and J. Bowen, “The Kynurenine Pathway and Kynurenine 3-Monooxygenase Inhibitors,” Molecules, vol. 27, no. 1, p. 273, 2022.
[84] R. Cerniway, R. Morrison, A. Byford, A. Lankford, J. Headrick, D. Van Wylen and G. Matherne, “A1 adenosine receptor overexpression decreases stunning from anoxia-reoxygenation: role of the mitochondrial K(ATP) channel,” Basic Res Cardiol, vol. 97, no. 3, pp. 232-238, 2002.
[85] M. Yamaga, H. Tani, A. Yamaki, T. Tatefuji and K. Hashimoto, “Metabolism and pharmacokinetics of medium chain fatty acids after oral administration of royal jelly to healthy subjects,” RCS Adv, vol. 9, no. 27, pp. 15392-15401, 2019.
[86] A. Sachdeva, S. Seth, A. Khosla and S. Sachdeva, “Study of some common biochemical bone turnover markers in postmenopausal women,” Indian J Clin Biochem, vol. 20, no. 1, pp. 131-134, 2005.

1. A method of modifying levels of a microbially-influenced metabolite in human serum generated by resistant potato starch fermentation in the human's gut comprising administering to said human an effective amount of a suitable resistant potato starch on a dosage regimen.

2. (canceled)

3. (canceled)

4. The method according to claim 1 wherein the microbially-influenced metabolite is selected from the group consisting of collagen, 5-hydroxylysine, 4-hydroxyproline, pregnenolone sulfate, 3-beta-hydroxy-5-cholestenoic acid, 3-hydroxykynurenine, histamine, N-acetyl putrescine, N-acetyl spermidine, acylcarnitine, carnitine, propionyl carnitine, serotonin, coenzyme Q10 and hydroxydecanoic acid.

5. (canceled)

6. The method according to claim 1 wherein the individual in need of such treatment has baseline serum hydroxylysine levels greater than 0.24 μmol/L.

7. The method according to claim 1 wherein the individual in need of such treatment has baseline serum hydroxyproline levels greater than 13 μmol/L.

8. The method according to claim 1 wherein the individual in need of such treatment has impaired collagen metabolism, has a hereditary condition that affects collagen synthesis, is elderly, or subjects their skin to environmental exposures that promote collagen degradation, including ultraviolet radiation.

9. (canceled)

10. (canceled)

11. The method according to claim 1 wherein the individual in need of such treatment has baseline serum hydroxylysine levels greater than 0.24 μmol/L.

12. (canceled)

13. (canceled)

14. (canceled)

15. (canceled)

16. (canceled)

17. (canceled)

18. (canceled)

19. The method according to claim 1 wherein the individual in need of such treatment has baseline serum pregnenolone sulfate levels less than 40 ng/mL.

20. The method according to claim 1 wherein the individual in need of such treatment has menstruation-related migraines or headaches.

21. (canceled)

22. The method according to claim 1 wherein the individual in need of such treatment is a student, is studying, or is in need of enhanced memory function.

23. (canceled)

24. The method according to claim 1 wherein the individual in need of such treatment has baseline serum 3-beta-hyrdoxy-5-cholestenoic acid levels <67 ng/mL.

25. The method according to claim 1 wherein the individual in need of such treatment has Alzheimer's disease or has a heredity risk of developing Alzheimer's disease.

26. The method according to claim 1 wherein the individual in need of such treatment requires a modulator of gamma secretase activity.

27. (canceled)

28. The method according to claim 1 wherein the individual in need of such treatment has dyslipidemia, high cholesterol, obesity, metabolic syndrome, diabetes, or a related metabolic disorder.

29. (canceled)

30. The method according to claim 1 wherein the individual in need of such treatment has dyslipidemia, high cholesterol, obesity, metabolic syndrome, diabetes, or a related metabolic disorder.

31. (canceled)

32. (canceled)

33. (canceled)

34. The method according to claim 1 wherein the individual in need of such treatment is also taking a probiotic.

35. (canceled)

36. (canceled)

37. (canceled)

38. The method according to claim 1 wherein the survival or viability of the probiotic are increased in an individual in need of such treatment.

39. The method according to claim 1 wherein the individual in need of such treatment has dysbiosis, elevated levels of proteobacteria, or low levels of Bifidobacterium, Lactobacillus, or Akkermansia.

40. The method according to claim 1 wherein the individual in need of such treatment is taking or has recently taken antibiotics.

41. The method according to claim 1 wherein the individual in need of such treatment suffers from digestive distress, diarrhea, constipation, or bowel movement inconsistency.

42. (canceled)

43. The method according to claim 1 wherein the individual in need of such treatment has baseline urine 3-hydroxykynurenine levels greater than 450 μg/g creatinine.

44. (canceled)

45. (canceled)

46. The method according to claim 1 wherein the individual in need of such treatment is receiving hydrocortisone treatment or is pregnant.

47. The method according to claim 1 wherein the individual in need of such treatment experiences elevated oxidative stress, has mitochondrial dysfunction, or is diabetic.

48. (canceled)

49. The method according to claim 1 wherein the individual in need of such treatment has baseline serum histamine levels greater than 10 nmol/L.

50. The method according to claim 1 wherein the individual in need of such treatment has inflammation, itching, swelling, an allergic reaction, or suffers from allergies.

51. The method according to claim 1 wherein the individual in need of such treatment has histamine intolerance and/or mast cell activation syndrome (MCAS).

52. The method according to claim 1 wherein the individual in need of such treatment has excessive intestinal permeability and/or ‘leaky gut’.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. The method according to claim 1 wherein the individual in need of such treatment has an elevated ratio of small-and medium-chain carnitines to free carnitine compared to a control individual.

71. (canceled)

72. The method according to claim 1 wherein the individual in need of such treatment has intestinal permeability due to non-steroidal anti-inflammatory drug (NSAID) use, chemotherapy, or other interventions that cause intestinal permeability.

73. (canceled)

74. The method according to claim 1 wherein the individual in need of such treatment has or is at genetic risk of developing intestinal permeability as a co-morbidity to irritable bowel disease or inflammatory bowel disease (e.g. Crohn's disease or colitis), diabetes, or as a primary affliction.

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. The method according to claim 1 wherein the individual in need of such treatment has baseline serum coenzyme Q10 levels less than 0.5 μmol/L.

101. (canceled)

102. The method according to claim 1 wherein the individual in need of such treatment is suffering from or predisposed to heart conditions, such as congestive heart failure, high blood pressure, or high cholesterol, has or is suspected of having diabetes or Parkinson's disease, or is taking statin drugs and has statin-induced myopathy.

103. (canceled)

104. (canceled)

105. The method according to claim 1 wherein the individual in need of such treatment is taking statin drugs and has statin-induced myopathy.

106. (canceled)

107. (canceled)
